(19) United States
(12) Patent Application Publication
(10) Pub. No.: US 2023/0310542 A1
(43) Pub. Date: Oct. 5, 2023

(54) EPIGENETIC MODIFIERS TO TREAT RETINAL DEGENERATIONS

(71) Applicant: The Penn State Research Foundation, University Park, PA (US)

(72) Inventors: Colin J. Barnstable, University Park, PA (US); Evgenya Y. Popova, University Park, PA (US); Joyce Tombran-Tink, Harrisburg, PA (US)

(21) Appl. No.: 18/295,484

(22) Filed: Apr. 4, 2023

Related U.S. Application Data
(60) Provisional application No. 63/327,307, filed on Apr. 4, 2022.

(51) Int. Cl. A61K 38/15; A61K 31/135; A61K 31/445; A61K 31/706; A61K 31/517; A61K 45/06; A61P 27/02

(52) U.S. Cl. CPC A61K 38/15; A61K 31/135; A61K 31/445; A61K 31/706; A61K 31/517; A61K 45/06; A61P 27/02

(57) ABSTRACT

The present disclosure relates to the prevention and/or treatment of retinal degeneration using epigenetic modifying agents and their uses thereof.

This application claims the benefit of U.S. Provisional Application No. 63/327,307, filed on Apr. 4, 2022, which is incorporated herein by reference in its entirety.

### STATEMENT REGARDING FEDERALLY SPONSORED RESEARCH

This invention was made with Government Support under Grant No. R21 EY029992 awarded by the National Institutes of Health. The Government has certain rights in the invention.

### REFERENCE TO SEQUENCE LISTING

The sequence listing submitted on Mar. 21, 2023, as an .XML file entitled “11196-080US1 ST.26sequence.xml” created on Mar. 31, 2023, and having a file size of 95,649 bytes is hereby incorporated by reference pursuant to 37 C.F.R. § 1.52(e)(5).

### FIELD

The present disclosure relates to the prevention and/or treatment of retinal degeneration using epigenetic modifying agents and their uses thereof.

### BACKGROUND

The epigenetic landscape of a cell defines certain patterns of gene expression, including transcriptional responses to a changing environment. There have been efforts to modify the epigenome both to channel normal development and to combat a variety of diseases. Currently, these efforts have been carried out on dividing cells, particularly tumor cells. However, the nervous system and closely associated tissues, such as the eye, presents a different substrate for epigenetic modification because these tissues are usually non-replicating after final differentiation. Because of the genetic heterogeneity of many retinal diseases and disorders, there has been limited progress in identifying suitable treatments for patients. Thus, targeting the epigenome of the retinal system can allow expression of genes promoting anti-inflammatory and immunomodulatory responses as well as promoting healthy visual functions.

Given limitations of treatments and therapies available for retinal diseases and disorders, there is need to develop gene modifying agents to treat and/prevent retinal diseases and disorders. The compositions and methods disclosed herein address these and other needs.

### SUMMARY

The present disclosure provides methods treating, preventing, or reducing a retinal disease or disorder in a subject.

In one aspect, disclosed herein are methods of treating, inhibiting, decreasing, reducing, ameliorating, and/or preventing a retinal disease or disorder (such as, for example, retinitis pigmentosa or macular degeneration) in a subject, the method comprising administering to the subject a composition comprising an epigenetic modifier and a pharmaceutically acceptable carrier, wherein the epigenetic modifier comprises an inhibitor of chromatin modifying enzymes.

Also disclosed herein are methods of treating, inhibiting, decreasing, reducing, ameliorating, and/or preventing a retinal disease or disorder of any preceding aspect, wherein the inhibitor comprises a demethylase inhibitor (such as, for example, a lysine-specific demethylase 1 (LSD1) inhibitor), a methyltransferase inhibitor (such as, for example a histone methyltransferase inhibitor), a deacetylase inhibitor (such as, for example, a histone deacetylase 1 (HDAC) inhibitor), or variants thereof. In some embodiments, the LSD1 inhibitor comprises tranylcypromine (TCP), GSK2879552, or variants thereof. In some embodiments, the histone methyltransferase inhibitor comprises 3-deazaneplanocin A (DZNep), UNC0642, or variants thereof. In some embodiments, the HDAC1 inhibitor comprises romidepsin, or variants thereof.

In one aspect, disclosed herein are methods of treating, inhibiting, decreasing, reducing, ameliorating, and/or preventing a retinal disease or disorder of any preceding aspect, wherein the composition is administered for at least 14 days.

Also disclosed herein are methods of treating, inhibiting, decreasing, reducing, ameliorating, and/or preventing a retinal disease or disorder of any preceding aspect, wherein the composition is administered by a method selected from the group consisting of administration as an eye drop, administration by an intraocular injection, administration as a gel to an eye of the subject, administration as an implant in the eye that releases the epigenetic modifier over time, administration as an expression vector that expresses the epigenetic modifier, and administration using a cell-based expression system. In some embodiments, the pharmaceutically acceptable carrier comprises a saline solution, a gelatin composition, an excipient, a diluent, a salt, a buffer, a stabilizer, a lipid, an emulsion, or a nanoparticle.

In one aspect, disclosed herein are methods of treating, inhibiting, decreasing, reducing, ameliorating, and/or preventing a retinal disease or disorder of any preceding aspect, wherein the method comprises administering an additional therapeutic agent to the subject, wherein the therapeutic agent comprises an antibiotic, an anesthetic, a sedative, an anti-inflammatory composition, or a hydrating solution. In some aspects, the additional therapeutic agent is comprised in the same composition as the epigenetic modifier. In some aspects, the additional therapeutic agent is comprised in a different composition from the epigenetic modifier.

Also disclosed herein are methods of treating, inhibiting, decreasing, reducing, ameliorating, and/or preventing a retinal disease or disorder of any preceding aspect, wherein the epigenetic modifier decondenses chromatin to increase or maintain expression of one or more genes selected from the group consisting of CRX, NRL, RHO, PRPH2, NR2E3, PDE6B, SAG, ROM1, CNGA1, CNGB1, NEUROD1, PTP4A3, ABCA4, FAM83G, LEFTY2, SFRP5, and UPK1B. In some embodiments, the epigenetic modifier alters the chromatin to decrease expression of one or more genes selected from the group consisting of GFAP, C1QB, C1QA, H2-AA, CX3CR1, PTPRC, CD74, CST7, and AIF1.

In one aspect, disclosed herein are methods of treating, inhibiting, decreasing, reducing, ameliorating, and/or preventing a retinal disease or disorder of any preceding aspect, wherein the method reduces or prevents degeneration of a retinal cell. In some embodiments, the method decreases inflammation, gliosis, or cell death in the subject. In some embodiments, the method increases an anti-inflammatory response in the subject.

Also disclosed herein are methods of treating, inhibiting, decreasing, reducing, ameliorating, and/or preventing a retinal disease or disorder of any preceding aspect, wherein the subject is a mammal. In some embodiments, the subject is a human.

### BRIEF DESCRIPTION OF FIGURES

The accompanying figures, which are incorporated in and constitute a part of this specification, illustrate several aspects described below.

FIGS. 1A, 1B, 1C, 1D, and 1E show the treatment of rd10 mice with inhibitors specific for LSD1 and HDAC1 leads to neuroprotection and preservation of rod photoreceptors. FIG. 1A shows the immunofluorescence microscopic images of sections of retinas from rd10 mice from PN15 to PN60 stained with RHO (green), OPN1SW (red), and nuclear counterstained with Hoechst33358 (blue); GCL, ganglion cell layer; INL, inner nuclear layer; ONL, outer nuclear layer. Scale bar=20 um. FIG. 1B shows the immunofluorescence microscopic images of retina sections from PN24 rd10 mice treated from PN9 till PN24 with inhibitors for HDAC1 (romidepsin) or LSD1 (TCP and GSK) or only saline (control), stained with RHO (green), OPN1SW (red), and nuclear counterstained with Hoechst33358. FIG. 1C shows the image quantification of immunofluorescence intensity for RHO was carried out for 4 biological and 3 technical replicates (±SEM) for the rd10 retinas treated with GSK or saline (control); **** p<0.0001 FIG. 1D shows the rods rows were counted in central retina for PN24 mice treated from PN9 till PN24 with inhibitors for LSD1 (TCP and GSK) and HDAC1 (romidepsin) or only with saline (WT and rd10) for 3-5 biological and 3 technical replicas (±SEM); ** p<0.01, **** p<0.0001. FIG. 1E shows the ONL thickness was measured in central retina for PN24 mice treated from PN9 till PN24 with inhibitors for HDAC1 (romidepsin), LSD1 (TCP and GSK) or only with saline (WT and rd10) for 3-5 biological and 3 technical replicates (±SEM) for each sample; *p<0.05, ** p<0.01, *** p<0.001, **** p<0.0001.

FIGS. 2A, 2B, 2C, 2D, 2E, and 2F show the continuous presence of LSD1 inhibitors is needed to prevent rod degeneration in a Retinitis Pigmentosa model. FIGS. 2A-2D show the immunofluorescence microscopic images of sections of rd10 mouse retinas treated with LSD1 inhibitor GSK at different time frames, stained with anti-H3K4me2 antibody (red) and nuclear counterstained with Hoechst33358 (blue). FIG. 2A shows the treatment with GSK from PN15 till PN24, assayed at PN24 and compared to controls treated with saline only. FIG. 2B shows the rd10 mice litter was treated with GSK from PN9 till PN17; half litter assayed at PN24, half assayed at PN17 and compared PN24 to PN17. FIG. 2C shows the rd10 mice litter was treated with GSK from PN9 till PN24: half litter assayed at PN24, half assayed at PN45 and compared PN45 to PN24. FIG. 2D shows the rd10 mice were treated with saline or with GSK each second day (ESD) from PN9 till PN24. FIG. 2E shows the rods rows were counted in central retina for rd10 mice for 3-5 biological and 3 technical replicas (±SEM); ** p<0.01, *** p<0.001, **** p<0.0001. Time frames correspond to FIG. 2A (pink/red bars); FIG. 2B (grey bars); FIG. 2C (yellow bars); FIG. 2D (green bars). FIG. 2F. Evaluation of visual function in rd10 mice treated from PN9 till the PN32 with saline (control) and GSK ESD. Spatial frequency (SF) threshold were assessed using a video camera to monitor optomotor reflex. SF was assessed at 100% contrast. The SF thresholds were identified as the highest values that elicited the reflexive head movement. SF (acuity) was measured for 6 eyes of rd10 control and 6 eyes for rd10 treated with GSK ESD on PN24, PN25, PN26, PN28 and PN32 and then averaged for each eye, ** p<0.01.

FIGS. 3A and 3B show the treatment of mice with inhibitors specific for LSD1 and HDAC1 slow gain of weight. FIG. 3A shows the weight of mice at PN24 after rd10 mice were treated from PN9 till PN24 with romidepsin, TCP, GSK1.5, GSK4.2 mg/kg and GSK4.2 each second day (ESD). FIG. 3B shows the WT mice were treated with saline, GSK, or TCP. Experiments were done for 3-5 biological replicas, *** p<0.001 **** p<0.0001 (±SEM).

FIGS. 4A, 4B, 4C, 4D, 4E, 4F, 4G, and 4H show the RNA-seq analysis of altered retinal gene expression under LSD1 inhibition. FIG. 4A shows the overall changes in number of genes up and down regulated in WT and rd10 mice treated by GSK and compare to saline treated controls (FDR<0.05; FC—greater than 2 or smaller than 0.5). RNA-seq was done for 2 WT, 3WT+GSK, 3 rd10 and 3 rd10+GSK retinal samples. FIG. 4B shows the heatmap of DEG simultaneously upregulated in rd10 and WT mice retinas under GSK treatment (p<0.05; FC—bigger than 1.75 or smaller than 0.8). FIG. 4C shows the heatmap of DEG between WT and rd10 retinas (FDR<0.05; FC—greater than 2 or smaller than 0.5). FIG. 4D shows the heatmap of DEG between rd10 and treated with GSK rd10 retinas (FDR<0.05; FC—greater than 2 or smaller than 0.5). FIG. 4E shows the top Ingenuity canonical pathways for DEG between WT and rd10 retinas (FDR<0.05; FC—greater than 2 or smaller than 0.5) according to IPA. FIG. 4F shows the top upstream regulators according to IPA for DEG between WT and rd10 retinas (FDR<0.05; FC—greater than 2 or smaller than 0.5). FIG. 4G shows the top Ingenuity canonical pathways for DEG between rd10 and treated with GSK rd10 retinas (FDR<0.05; FC—greater than 2 or smaller than 0.5) according to IPA. FIG. 4H shows the top upstream regulators according to IPA for DEG between rd10 and treated with GSK rd10 retinas (FDR<0.05; FC—greater than 2 or smaller than 0.5). In all panels orange color represents upregulated gene or activated pathway; blue color represents downregulated gene or inhibited pathway.

FIGS. 5A, 5B, 5C, 5D, 5E, 5F, 5G, 5H, 5I, 5J, 5K, and 5L show the treatment of mouse model of Retinitis Pigmentosa with inhibitors specific for LSD1 and HDAC1 leads to preservation of expression of rod-specific genes. FIG. 5A shows the heat map of expression of different group of rod-specific genes measured by RT-PCR for retinas from PN24 rd10 (or WT) mice treated from PN9 till PN24 with inhibitors for LSD1 (TCP and GSK) and HDAC1 (romidepsin) compared to controls rd10 (or WT) mice treated with saline only for 3-5 biological and 3 technical replicas (±SEM). The relative expression level for each gene was calculated by the 2-ΔΔCt method and normalized to GAPDH; * p<0.05; ** p<0.01; *** p<0.001, ****p<0.0001 with fold increase in orange or decrease in green. FIGS. 5B-5K shows the comparison of rod specific gene expression levels in mice PN24 retina of WT mice and rd10 mice, treated with saline, or in rd10 treated with GSK from PN9 till PN24. Experiments were done for 4 biological and 3 technical replicas, ** p<0.01; *** p<0.001, ****p<0.0001. FIG. 5L shows the increasing PDE6B protein level. Anti-PDE6B Western blot with representative samples of retina at PN24 from WT mice treated with saline and rd10 mice treated with saline or with GSK from PN9 till PN24. Histone H4 Coomassie staining was used as loading control. Band intensity quantification was done using 3-5 biological and 3 technical replicates for anti-PDE6B Western. For each sample, anti-PDE6B bands intensity was normalized to the average of quantified intensities of Coomassie bands for histone H4 and anti-ACTB Western band.

FIGS. 6A, 6B, 6C, 6D, 6E, and 6F show the changes in expression of retina gene markers are closely correlate with rod preservation if alternative time windows were used for i.p. injection of epigenetic inhibitors. Heat map of expression of different group of genes measured by RT-PCR and rod rows counted in central retina for retinas from mice treated with inhibitors for LSD1 (TCP and GSK) for 3-5 biological and 3 technical replicas (±SEM); **p<0.01, **** p<0.0001. The relative expression level for each gene was calculated by the 2-ΔΔCt method and normalized to GAPDH; * p<0.05; ** p<0.01; *** p<0.001, ****p<0.0001 with fold increase in orange or decrease in green. Column A: rd10 mice were treated with GSK from PN9 till PN24 each second day, assayed at PN24 and compared to controls treated with saline only. Column B: rd10 mice were treated with TCP from PN10 till PN29, assayed at PN40 and compared to controls treated with saline only. Column C: rd10 mice were treated with GSK from PN15 till PN24, assayed at PN24 and compared to controls treated with saline only. Column D: rd10 mice litter was treated with GSK from PN9 till PN17; half litter assayed at PN24, half assayed at PN17 and compared PN24 to PN17. Column E: rd10 mice litter was treated with GSK from PN9 till PN24: half litter assayed at PN24, half assayed at PN45 and compared PN45 to PN24.

FIGS. 7A, 7B, 7C, 7D, 7E, 7F, 7G, 7H, and 7I show the Effect of treatment of mouse model of Retinitis Pigmentosa with inhibitors of LSD1 and HDAC1 on other retina cell types.

FIG. 7A shows the heat map of expression of different groups of retina genes measured by RT-PCR for retinas from PN24 mice treated from PN9 till PN24 with inhibitors for LSD1 (TCP and GSK) and HDAC1 (romidepsin) compared to control rd10 mice treated with saline only for 3-5 biological and 3 technical replicates (±SEM). The relative expression level for each gene was calculated by the 2-ΔΔCt method and normalized to GAPDH; * p<0.05; ** p<0.01; *** p<0.001, ****p<0.0001 with fold increase in orange or decrease in green. FIGS. 7B-7I show the comparison of cone photoreceptor specific gene expression levels in PN24 retina of WT mice, rd10 mice treated with saline, or rd10 treated with GSK from PN9 till PN24. Data shown for 4 biological and 3 technical replicates, * p<0.05; *** p<0.001, ****p<0.0001. Comparison of expression levels for cone genes in mouse retina at PN24; WT mice were treated with saline and rd10 mice were treated with saline or with GSK ESD from PN9 till PN24. Experiments were done for 3-4 biological and 3 technical replicates, *p<0.05, ** p<0.01, *** p<0.001 (±SEM).

FIGS. 8A, 8B, 8C, 8D, and 8E show the GSK treatment is not harmful for cells in INL. Comparison of gene expression levels in rd10 mice PN40 retina treated with saline (control) or with GSK from PN30 till PN40. Experiments were done for 5 biological and 3 technical replicates, *p<0.05; **** p<0.0001(±SEM).

FIGS. 9A, 9B, 9C, 9D, 9E, 9F, 9G, 9H, 9I, 9J, 9K, 9L, 9M, 9N, 90, 9P, and 9Q show the treatment of rd10 with inhibitors specific for LSD1 and HDAC1 leads to decreased cell death, gliosis, and inflammation. FIG. 9A shows the heat map of expression of cell death genes measured by RT-PCR for retinas PN24 mice treated from PN9 till PN24 with inhibitors for LSD1 (TCP and GSK) and HDAC1 (romidepsin) compared to controls rd10 mice treated with saline only. The relative expression level for each gene was calculated by the 2-ΔΔCt method and normalized to GAPDH; with fold increase shown in orange or decrease shown in green. FIG. 9B shows the immunofluorescence microscopic images of retina sections from PN24 WT mice treated from PN9 till PN24 with TCP or only saline (control), stained with TUNEL and nuclear counterstained with Hoechst33358. GCL, ganglion cell layer; INL, inner nuclear layer; ONL, outer nuclear layer. FIG. 9C shows the immunofluorescence microscopic images of retina sections from PN24 rd10 and WT mice treated from PN9 till PN24 with GSK, romidepsin or only saline (control), stained with IBA1 (green) (for AifI gene), GFAP (red), and nuclear counterstained with Hoechst33358. GCL, ganglion cell layer; INL, inner nuclear layer; ONL, outer nuclear layer. Scale bar=20 um White arrowheads point out IBA1 positive microglia cells in ONL. FIGS. 9D-9Q show the comparison of gene expression levels for Gfap in mice at PN24; WT and rd10 mice were treated with saline or with GSK from PN9 till PN24. Image quantification of immunofluorescence intensity from FIG. 9C for GFAP for the rd10 and WT retinas treated with GSK or saline (control). Comparison of gene expression levels for inflammatory markers in mice at PN24; rd10 mice were treated with saline or with romidepsin from PN9 till PN24. Comparison of gene expression levels for inflammatory markers in mice at PN24; rd10 mice were treated with saline or with GSK from PN9 till PN24. In all cases experiments were carried out with 3-4 biological and 3 technical replicates, * p<0.05; ** p<0.01; *** p<0.001; **** p<0.0001(±SEM).

FIGS. 10A, 10B, 10C, 10D, and 10E show the LSD1 inhibition promotes epigenetic changes that create more open and accessible chromatin in rod nuclei. FIG. 10A shows the ONL thickness and rods rows were counted in central retina for PN24 WT mice treated from PN9 till PN24 with TCP or only with saline for 3-5 biological and 3 technical replicas (±SEM); ** p<0.01. FIG. 10B shows the comparison of number of foci in rod nuclei in central retina at PN24 for WT mice treated with saline or TCP and rd10 mice treated with saline or GSK from PN9 till PN24. Experiments were done for 3-4 biological and 3 technical replicas; 300-450 nuclei were counted for each sample. Left panels. Representative immunofluorescence microscopic image of rod photoreceptors in ONL in central mouse retina sections from PN24 rd10 mice treated with GSK from PN9 till PN24. Bottom panel: anti-H3K4me2 staining is in green (euchromatin), anti-H3K9me2 staining is in red (facultative heterochromatin) and nuclear counterstaining with Hoechst33358 in blue (constitutive heterochromatin). Upper panel: image of only Hoechst33358 counterstaining demonstrates that rod photoreceptor nuclei have 1, 2 or 3 foci of heterochromatin. Right panels: Comparison of number of foci in rod nuclei in central retina at PN24 for WT mice treated with saline or TCP and rd10 mice treated with saline or GSK from PN9 till PN24. Experiments were done for 3-4 biological and 3 technical replicas; 300-450 nuclei were counted for each sample. FIG. 10C shows the left panel: Anti-H3K4me2 and anti-H3K9me2 Western blot with samples of retina at PN24 from WT mice treated with saline or with GSK from PN9 till PN24. Histone Coomassie stating was used as loading control. Right panels: Band intensity quantification was done for 3 biological and 3 technical replicates for anti-H3K4me2 and H3K9me2 Western. The intensity of bands for histone modifications were normalized on intensity for core histone Coomassie staining. FIG. 10D shows the heat map of expression of progenitor/cell cycle genes measured by RT-PCR for retinas from PN24 mice WT treated from PN9 till PN24 with inhibitors for LSD1 (TCP and GSK) relative to WT treated with saline; and rd10 mice treated from PN9 till PN24 with GSK and compared to rd10 treated with saline. Each set of data represents 3-5 biological and 3 technical replicates (±SEM). The relative expression level for each gene was calculated by the 2-ΔΔCt method and normalized to GAPDH; *p<0.05, ** p<0.01, *** p<0.001, **** p<0.0001 with fold increase in orange or decrease in green. FIG. 10E shows the comparison of H3K4me2 and H3K9me2 accumulation on gene regulatory elements such as promoter and enhancer in mice retina at PN24; WT mice were treated with saline or GSK from PN9 till PN24. Quantitative PCRs were done with primers (Table 2) for area around gene regulatory elements in 3 technical replicas.

FIGS. 11A and 11B show the EZH2 enzyme participates in methylation of H3K27me3 and its inhibitor DZNep shows similar effects to GSK2879552 (see figure) in rd10 mice.

FIGS. 12A, 12B, and 12C show the G9a/GLP enzyme participates in methylation of H3K9me2. Inhibiting this enzyme with UNC0642 has a similar effect in preserving rod photoreceptors.

FIGS. 13A, 13B, 13C, 13D, 13E, 13F, 13G, and 13H show the GSK2879552 was administered directly to the eye. A solution of GSK2879552 was prepared in artificial tears and applied daily to both eyes of rd10 mice in a volume of 5 μl. Several rod specific genes were upregulated in treated animals.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inhibitors” or “antagonist” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists. Control samples (untreated with inhibitors) are assigned a relative activity value of 100%. Inhibition of a described target protein is achieved when the activity value relative to the control is about 80%, optionally 50% or 25, 10%, 5%, or 1% or less.

A “variant” or a “derivative” of a particular inhibitor may be defined as a chemical or molecular compound having at least 50% identity to a parent or original inhibitor. In some embodiments a variant inhibitor may show, for example, at least 60%, at least 70%, at least 80%, at least 90%, at least 91%, at least 92%, at least 93%, at least 94%, at least 95%, at least 96%, at least 97%, at least 98%, or at least 99% or greater identity relative to a reference parent or original inhibitor.

By “reduce” or other forms of the word, such as “reducing” or “reduction,” means lowering of an event or characteristic (e.g., vision loss). It is understood that this is typically in relation to some standard or expected value, in other words it is relative, but that it is not always necessary for the standard or relative value to be referred to. For example, “reduces degeneration or vision loss” means reducing the rate of degeneration of a tissue or reducing the rate of vision loss”.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

As used herein, “preserve,” “preserved,” “preservation,” “preserving” and any grammatical variations thereof as used herein, refers to the act of keeping any object, composition, or compound intact, alive, or free from decomposition/decay.

A “gene” refers to a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated.

The terms “treat,” “treating,” and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the disclosure may be applied preventively, prophylactically, palliatively, or remedially. Treatments are administered to a subject prior to onset (e.g., before obvious signs of degeneration), during early onset (e.g., upon initial signs and symptoms of degeneration), or after an established development of degeneration.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN (ICI, Inc.; Bridgewater, New Jersey), polyethylene glycol (PEG), and PLURONICS (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

“Tissue degeneration” or “degeneration” refers to the process by which tissue deteriorates and loses its functional ability due to genetic mutations, traumatic injury, aging, or wear and tear.

A “chromosome” refers to a long DNA molecule comprising part or all of the genetic material of an organism. Most chromosomes comprise very long thin DNA strands coated with packaging proteins, including but not limited to histone proteins and other chaperone proteins, critical for binding, modifying, remodeling, and/or condensing/decondensing the DNA strands into the tightly compacted chromosome structures. Such chromosomes are formed to maintain and preserve genetic stability and integrity.

“Chromatin” refers to a complex of DNA and protein generally found in eukaryotic cells whose primary function is to package long DNA molecules into more compact, denser structures. This prevents the DNA strands from becoming tangled and plays additional roles in reinforcing the DNA during cell division, preventing DNA damage, and regulating gene expression and DNA replication. The primary protein components of chromatin are histone proteins comprising an octamer of two sets of four histone core proteins (Histone H2A, Histone H2B, Histone H3, and Histone H4) binding to DNA and function as anchors around which the strands are wound. In general, there are three levels of chromatin organization: 1) DNA wraps around histone proteins, forming nucleosomes also referred to as the “beads on a string” structure; 2) Multiple histones wrap into small (about 30 nanometer long) fiber comprising nucleosome arrays in the most compact form; and 3) Higher-level DNA supercoiling of the small fibers of structure 2) to form the final chromosomal structure.

As used herein, “epigenetic modification” refers to the heritable genetic changes the affect gene expression activity without altering the DNA or RNA sequence. These genetic changes include but are not limited to DNA or RNA methylation and histone modifications (i.e.: methylation and/or acetylation) that alter DNA or RNA accessibility and structure, thereby regulating gene expression patterns.

The “retina” is the innermost, light-sensitive layer of tissue within the eye of most vertebrates, including, but not limited to humans. Retinal tissue comprises several layers made up of light-sensing cells called photoreceptor cells, which detect and process light coming into the retina. The “macula” refers to an oval-shaped pigmented area in the center of the retina of most vertebrate eyes, including, but not limited to humans. This area of the retina is responsible for producing central, high-resolution color vision. High-resolution color vision is lost when the macula is damaged as a result of macular degeneration. The “fovea” refers to the more centrally located region within the macula of the retina of most vertebrates, including, but not limited to humans. The fovea is a small, central locus of densely packed photoreceptor cells, called cones, responsible for sharp, central vision.

Methods of Treating and/or Preventing a Retinal Disease or Disorder

In a broad sense, epigenetics refers to a bridge between genotype and phenotype, wherein changes are made to a locus or final chromosome structure without altering the underlying DNA sequence of an individual. In more specific terms, epigenetics is described as a study of any potentially stable and, usually, heritable change in gene expression or cellular phenotype that occurs without physical changes to the nucleotides within a DNA sequence. Generally, epigenetic regulation requires altering chromosome structure to influence availability of specific DNA sequences to allow for gene expression and regulation of transcription. The chromosome is a compaction of a long DNA sequence, which causes some DNA sequences limited exposure for access to transcriptional machinery. However, modifications, including, but not limited to methylation and acetylation, of chromosomal proteins called histone, leads to decondensation, or unraveling, of the chromosome to allow for gene expression.

In general, there are four histone modifying enzymes that also impact epigenetics, 1) Histone Methyltransferases (HMTs), 2) Histone Demethylases (HDMTs), 3) Histone Acetyltransferases (HATs), and 4) Histone Deacetylases (HDACs). The process of methylation is regulated by HMTs and HDMTs, wherein HMTs incorporate or add a chemical methyl group to histone proteins, whereas HDMTs remove said methyl group from histone proteins. Likewise, for acetylation, HATs incorporate or add a chemical acetyl group to histone proteins, whereas HDACs removes said acetyl group. Because epigenetics significantly impacts gene regulation and expression, it has become an attractive field for developing therapeutics and treatment methods for various diseases and disorders, especially with underlying genetic defects.

Retinal degeneration is an ocular condition characterized by partial or complete vision loss. Specifically, in cases of retinal degeneration, the retinal cells, also referred to as photoreceptor cells (cones or rods), are irreversibly damaged. Retinal diseases and disorders caused by retinal degeneration are complex conditions comprising heterogeneous genetic mutations and defects. In addition, there are currently few treatment options to prevent retinal degeneration and subsequent vision loss. Thus, relying on epigenetic-related treatments options to treat retinal diseases and/or disorders seeks to remedy these limitations.

In one aspect, disclosed herein are methods of treating, inhibiting, decreasing, reducing, ameliorating, and/or preventing a retinal disease or disorder (such as, for example, retinitis pigmentosa or macular degeneration), the method comprising administering to the subject a composition comprising an epigenetic modifier and a pharmaceutically acceptable carrier, wherein the epigenetic modifier comprises an inhibitor of chromatin modifying enzymes.

In some embodiments, the inhibitor comprises a demethylase inhibitor (such as, for example, a lysine-specific demethylase 1 (LSD1) inhibitor), a methyltransferase inhibitor (such as, for example a histone methyltransferase inhibitor), a deacetylase inhibitor (such as, for example, a histone deacetylase 1 (HDAC) inhibitor), or variants thereof. In some embodiments, the inhibitor comprises an acetyltransferase inhibitor, or variant thereof. In some embodiments, the epigenetic modifier comprises any combination of inhibitors comprising a demethylase inhibitor, a methyltransferase inhibitor, a deacetylase inhibitor, an acetyltransferase inhibitor, or variants thereof.

As noted above, in some embodiments, the demethylase inhibitor comprises a lysine-specific demethylase 1 (LSD1) inhibitor (such as, for example, tranylcypromine (TCP), GSK2879552, or variants thereof). In some embodiments, the LSD1 inhibitor is a natural LSD1 inhibitor or a non-natural LSD1 inhibitor. In some embodiments, a non-natural LSD1 inhibitor includes, but is not limited to ORY1001 (also referred to as RG6016 or Iadademstat), IMG7289 (also referred to as Bomedemstat), INCB059872, ORY2001 (Vafidemstat), CC90011, SP2577 (Seclidemstat), or variants thereof. In some embodiments, a natural LSD1 inhibitor includes, but not limited to protoberberine alkaloids (including, but not limited to epiberberine, columbamine, jatrorrhizine, berberine, and palmatine), flavones (including, but not limited to oroxylin A, skullcap flavone II, wogonin, wogonoside, baicalein, baicalin, hesperetin, hesperetin-7-O-glucoside, hesperidin, quercetin, isoquercetin, diosmetin, rutin, diosmetin-7-O-glucoside, diosmin, icaritin, icariin, and icariside II), diterpenoids (including, but not limited to geranylgeranoic acid (GGA), farnesol, oleacin, and tetrahydrofolate), curcumin, xanthones (including, but not limited to alpha-mangostin), stilbene, resveratrols (resveratrol-4e, resveratrol-8c, and variations thereof), secoiridoid, indole, phenols, polymyxin B, polymyxin E, and melatonin.

In some embodiments, the histone methyltransferase inhibitor comprises 3-deazaneplanocin A (DZNep), UNC0642, or variants thereof. In some embodiments, the histone methyltransferase inhibitor includes, but is not limited to MM-102, BIX01294, UNC0638, Chaetocin, EZH2, Sinefungin, and Pinometostat.

In some embodiments, the deacetylase inhibitor comprises a histone deacetylase 1 (HDAC) inhibitor. In some embodiments, the HDAC1 inhibitor comprises romidepsin, or variants thereof. In some embodiments, the HDAC1 inhibitor is a natural HDAC1 inhibitor or a non-natural HDAC1 inhibitor. In some embodiments, the non-natural HDAC1 inhibitor includes, but is not limited to Vorinostat, Tucidinostat, Panobinostat, Belinostat, Entionstat, Tacedinaline, Mocetinostat, Trapoxin B, Abexinostat, Scriptaid, C1994, MC1293, Parthenolide, KD5170, TC-H106, JNJ26481585, PC124781, Pimelic Diphenylamide 106, and pyroamide. In some embodiments, the natural HDAC1 inhibitor includes, but is not limited to phenylbutyrate and valproic acid.

In some embodiments, the composition or epigenetic modifier is administered for at least 14 days. In some embodiments, the composition or epigenetic modifier is administered for 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365 or more days, 13, 14, 15, 16, 17, 18, 19, 20, 21, 22, 23 months, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 years. In some aspects, the epigenetic modifier is administered for the remaining life of the subject.

In some embodiments, the composition or epigenetic modifier is administered daily. In some embodiments, the composition or epigenetic modifier is administered every day, every 2 days, every 3 days, every 4 days, every 5 days, every 6 days, every 7 days, or more. In some embodiments, the composition or epigenetic modifier is administered every week, every 2 weeks, every 3 weeks, every 4 weeks, or more. In some embodiments, the composition or epigenetic modifier is administered every month, every 2 months, every 3 months, every 4 months, every 5 months, every 6 months, every 7 months, every 8 months, every 9 months, every 10 months, every 11 months, every 12 months, or more. In some embodiments, the composition or epigenetic modifier is administered every year, every 2 years, every 3 years, every 4 years, every 5 years, or more.

In some embodiments, the composition or epigenetic modifier is administered by a method selected from the group consisting of administration as an eye drop, administration by an intraocular injection, administration as a gel to an eye of the subject, administration as an implant in the eye that releases the epigenetic modifier over time, administration as an expression vector that expresses the epigenetic modifier, and administration using a cell-based expression system. In some embodiments, the composition or epigenetic modifier is administered using a virus vector.

In some embodiments, the expression vector or cell-based expression system includes, but is not limited to a plasmid, a virus, and viral vector. A plasmid or a viral vector can be capable of extrachromosomal replication or, optionally, can integrate into the host genome. As used herein, the term “integrated” used in reference to an expression vector (e.g., a plasmid or viral vector) means the expression vector, or a portion thereof, is incorporated (physically inserted or ligated) into the chromosomal DNA of a host cell. As used herein, a “viral vector” refers to a virus-like particle containing genetic material which can be introduced into a eukaryotic cell without causing substantial pathogenic effects to the eukaryotic cell. A wide range of viruses or viral vectors can be used for transduction but should be compatible with the cell type the virus or viral vector are transduced into (e.g., low toxicity, capability to enter cells). Suitable viruses and viral vectors include adenovirus, lentivirus, retrovirus, among others. In some embodiments, the expression vector encoding a chimeric polypeptide is a naked DNA or is comprised in a nanoparticle (e.g., liposomal vesicle, porous silicon nanoparticle, gold-DNA conjugate particle, polyethyleneimine polymer particle, cationic peptides, etc.).

In some embodiments, the method comprises administering an additional therapeutic agent (i.e., a therapeutic agent that is not the epigenetic modifier) to the subject, wherein the therapeutic agent comprises an antibiotic, an anesthetic, a sedative, an anti-inflammatory composition, or a hydrating solution. It is noted that the additional therapeutic agent can be administered before, during, or after administration of the epigenetic modifier and the pharmaceutically acceptable carrier. It is also noted that the additional therapeutic agent can be administered one or more times as prescribed by a medical practitioner. In some embodiments, the additional therapeutic agent is administered 1, 2, 3, 4, 5, 6, 7, 8, 9, 10, or more times to the subject, as prescribed by a medical practitioner.

In some embodiments, the method comprises administering an additional antibiotic including, but not limited to penicillins (including, but not limited to amoxicillin, clavulanate and amoxicillin, ampicillin, dicloxacillin, oxacillin, and penicillin V potassium), tetracyclins (including, but not limited to demeclocycline, doxycycline, eravacycline, minocycline, omadacycline, sarecycline, and tetracycline), cephalosporins (cefaclor, cefadroxil, cefdinir, cephalexin, cefprozil, cefepime, cefiderocol, cefotaxime, cefotetan, ceftaroline, cefazidme, ceftriaxone, and cefuroxime), quinolones (also referred to as fluoroquinolones include, but are not limited to ciprofloxacin, delafloxacin, levofloxacin, moxifloxacin, and gemifloxacin), lincomycins (including clindamycin and lincomycin), macrolides (including, but not limited to azithromycin, clarithromycin, erythromycin, and fidaxomicin (ketolide)), sulfonamides (including sulfamethoxazole and trimethoprim, and sulfasalazine), glycopeptides (including, but not limited to dalbavancin, oritavancin, telavancin, and vancomycin), aminoglycosides (including, but not limited to gentamicin, tobramycin, and amikacin), carbapenems (including, but not limited to imipenem and cilastatin, meropenem, and ertapenem), and topical antibiotics (including, but not limited to neomycin, bacitracin, polymyxin B, and praxomine) used alone or in combination.

In some embodiments, the method comprises administering an additional non-steroidal anti-inflammatory compound including, but is not limited to aspirin, ibuprofen, ketoprofen, and naproxen. In some embodiments, the method comprises administering an additional anesthetic including, but is not limited to chloroprocaine, procaine, tetracaine, lidocaine, bupivacaine, ropivacaine, mepivacaine, and levobupivacaine. In some embodiments, the method comprises administering an additional sedative including, but is not limited to barbiturates, benzodiazepines, nonbenzodiazepines hypnotics, antihistamines, muscle relaxants, opioids, and methaqualone, or derivatives thereof.

It is understood and herein contemplated that the epigenetic modifier and the additional therapeutic agent can be administered in the same composition or in different compositions. Accordingly, it is further understood that when comprised in separate compositions, the additional therapeutic agent can be administered before, after, or concurrently with the epigenetic modifier.

In some embodiments, the method comprises administering an additional hydrating solution including, but not limited to a buffered solution comprising physiological concentration of salts, sugars, pH, and other compositions. In some embodiments, the method comprises administering an additional saline solution or saline buffer.

In some embodiments, the epigenetic modifier decondenses a chromatin to increase or maintain expression of one or more genes selected from the group consisting of CRX, NRL, RHO, PRPH2, NR2E3, PDE6B, SAG, ROM1, CNGA1, CNGB1, NEUROD1, PTP4A3, ABCA4, FAM83G, LEFTY2, SFRP5, and UPK1B. In some embodiments, the epigenetic modifier alters the chromatin to decrease expression of one or more genes selected from the group consisting of GFAP, C1QB, C1QA, H2-AA, CX3CR1, PTPRC, CD74, CST7, and AIF1.

In some embodiments, the epigenetic modifier decondenses a chromatin to increase, decrease, or maintain expression of one or more genes including, but not limited to IFNG, TNF, KDM1A, SAMD11, EMC1, DHDDS, POMGNT1, RPE65, CLCC1, PRPF3, ENSA, SEMA4A, CRB1, ADIPOR1, NEK2, FLVCR1, USH2A, AGBL5, ZNF513, IFT172, PCARE, FAM161A, SNRNP200, MERTK, CERKL, SAG, SPP2, TRNT1, MAPKAPK3, PROS1, ARL6, IMPG2, CLRN1, SLC7A14, PDE6B, CC2D2A, PROM1, GPR125, RP29, LRAT, CYP4V2, CWC27, P005, PDE6A, MAK, TULP1, GUCA1B, PRPH2, EYS, IMPG1, RP63, AHR, KLHL7, RP9, IMPDH1, KIAA1549, RP1L1, HGSNAT, RP1, TTPA, C8orf37, TOPORS, PRPF4, RPB, EXOSC2, RBP3, HK1, RGR, ARL3, ZNF408, BEST1, ROM1, MVK, RP16, RDH11, RDH12, TTC8, NR2E3, RLBP1, GNPTG, IFT140, RP22, BBS2, ARL2BP, CNGB1, DHX38, PRPF8, CA4, PRCD, FSCN2, PDE6G, REEP6, ARHGEF18, CRX, PRPF31, IDH3B, PANK2, KIZ, KIF3B, PRPF6, OFD1, RP6, RPGR, RP15, RP2, PGK1, PRPS1, RP24, RP34, MTATP6, MT-TS2, and MT-TP.

In some embodiments, the one or more genes are rod-specific genes. In some embodiments, the one or more genes are neuroprotective genes.

In some embodiments, the pharmaceutically acceptable carrier comprises a saline solution, a gelatin composition, an excipient, a diluent, a salt, a buffer, a stabilizer, a lipid, an emulsion, or a nanoparticle. One or more active agents (e.g. a HMT, a HDMT, a HAT, or a HDAC inhibitor) can be administered in the “native” form or, if desired in the form of salts, esters, amides, prodrugs, or a derivative that is pharmacologically suitable. Salts, esters, amides, prodrugs, and other derivatives of the active agents can be prepared using standards procedures known to those skilled in the art of synthetic organic chemistry and described, for example, by March (1992) Advanced Organic Chemistry; Reactions, Mechanisms, and Structure, 4th Ed. N.Y. Wiley-Interscience.

The composition may be administered in such amounts, time, and route deemed necessary in order to achieve the desired result. The exact amount of the composition will vary from subject to subject, depending on the species, age, and general condition of the subject, the severity of the degeneration, the particular composition, its mode of administration, its mode of activity, and the like. The composition is preferably formulated in dosage unit form for ease of administration and uniformity of dosage. It will be understood, however, that the total daily usage of the composition will be decided by the attending physician within the scope of sound medical judgment. The specific therapeutically effective dose level for any particular subject will depend upon a variety of factors including the retinal disease or disorder being treated and the severity of the degeneration; the activity of the composition employed; the specific inhibitor composition employed; the age, body weight, general health, sex and diet of the patient; the time of administration, route of administration, and rate of excretion of the specific composition employed; the duration of the treatment; drugs used in combination or coincidental with the specific composition employed; and like factors well known in the medical arts.

The exact amount of composition required to achieve a therapeutically or prophylactically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

The concentration of active agent(s) can vary widely and will be selected primarily based on activity of the active ingredient(s), body weight and the like in accordance with the particular mode of administration selected and the patient's needs. Concentrations, however, will typically be selected to provide dosages ranging from about 0.001 mg/kg/day to about 0.1 mg/kg/day, or 0.1 mg/kg/day to about 50 mg/kg/day, or higher doses. It will be appreciated that such dosages may be varied to optimize a therapeutic regimen in a particular subject or group of subjects.

In some embodiments, the composition can be prepared as a “concentrate,” e.g. in a storage container of a premeasured volume and/or a predetermined amount ready for dilution, or in a soluble capsule ready for addition to a specified volume of water, saline, or other diluent.

In some embodiments, the method reduces or prevents degeneration of a retinal cell. In some embodiments, the method reduces or prevents degeneration of a photoreceptor cell. In some embodiments, the method reduces or prevents degeneration of a rod photoreceptor cell. In some embodiments, the method reduces or prevents degeneration of a cone photoreceptor cell. In some embodiments, the method decreases inflammation, gliosis, or cell death in the subject. In some embodiments, the method increases an anti-inflammatory response in the subject.

In some embodiments, the retinal disease comprises retinitis pigmentosa. In some embodiments, the retinal disease comprises macular degeneration. In some embodiments, the retinal disease or disorder includes, but are not limited to rod-cone dystrophy, age-related macular degeneration, diabetic retinopathy, retinal tear, retinal detachment, macular hole, retinoblastoma, choroideremia, Stargardt disease, cone-rod dystrophy, Leber congenital amaurosis, Best vitelliform macular dystrophy, non-proliferative retinopathy, proliferative retinopathy, diabetic macular edema, cellophane maculopathy, central vein occlusion, branch retinal vein occlusion, macular pucker, degenerative myopia, lattice degeneration, retinal artery occlusion, branch vein occlusion, intraocular tumors, inherited retinal disorders, penetrating ocular traumas, pediatric and neonatal retinal disorders and/or diseases (including, but not limited to retinopathy of prematurity, juvenile macular degeneration, retrolental fibroplasia, macula disease, sickle cell retinopathy, colobomas, ocular toxocariasis, and TORCH syndrome (also referred to a group of infectious agents comprising (T)oxoplasmosis, (O)ther agents, (R)ubella (or German measles), (C)ytomegalovirus, and (H)erpes simplex virus present in prenatal and neonatal subject), cytomegalovirus (CMV) retinal infection, macula edema, uveitis, infectious retinitis, central serous retinopathy, endophthalmitis, hypertensive retinopathy, retinal hemorrhage, and solar retinopathy.

In some embodiments, the subject is a mammal. In some embodiments, the subject is human. In some embodiments, the subject is a non-human primate, bovine, equine, porcine, canine, feline, guinea pig, or a rodent.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Inhibition of Epigenetic Modifiers LSD1 and HDAC1 Blocks Rod Photoreceptor Death in Mouse Models in Retinitis Pigmentosa

Epigenetic modifiers are increasingly being investigated as potential therapeutics to modify and overcome disease phenotypes. Diseases of the nervous system present a particular problem as neurons are postmitotic and demonstrate relatively stable gene expression patterns and chromatin organization. The ability of epigenetic modifiers to prevent degeneration of rod photoreceptors in a mouse model of retinitis pigmentosa (RP), using rd10 mice of both sexes, was explored in this example. The histone modification eraser enzymes LSD1 and HDAC1 are known to have dramatic effects on the development of rod photoreceptors. In the RP mouse model, inhibitors of these enzymes blocked rod degeneration, preserved vision, and affected the expression of multiple genes including maintenance of rod-specific transcripts and downregulation those involved in inflammation, gliosis, and cell death. The neuroprotective activity of LSD1 inhibitors includes two pathways. First, through targeting histone modifications, they increase accessibility of chromatin and upregulate neuroprotective genes, such as from WNT pathway. Second, through non-histone targets, they inhibit transcription of inflammatory genes and inflammation. This process is going in microglia and lack of inflammation keeps rod photoreceptor alive.

Retinal degenerations are a leading cause of vision loss. RP is genetically very heterogeneous and the multiple pathways leading to cell death are one reason for slow progress in identifying suitable treatments for patients. Here it is demonstrated that inhibition of LSD land HDAC1 in a mouse model of RP leads to preservation of rod photoreceptors and visual function, retaining of expression of rod-specific genes, with decreased inflammation, cell death and Muller cell gliosis. It is contemplated that these epigenetic inhibitors cause more open and accessible chromatin, allowing expression of a neuroprotective genes. A second mechanism that allows rod photoreceptor survival is suppression of inflammation by epigenetic inhibitors in microglia. Manipulation of epigenetic modifiers is a new strategy to fight neurodegeneration in RP.

The retina continues to be a valuable model for studies of the role of the epigenome in both normal and pathophysiological conditions. Dynamic regulation of photoreceptor gene expression in the retina is governed not only by an array of specific transcription factors, but also by changing patterns of epigenetic regulation through histone modifications and resulting changes in overall chromatin structure. Two major classes of histone modifications are methylation and acetylation of lysine residues in the histone “tail” (N-terminal non-helical region). Methylation is controlled by two antagonist sets of enzymes, Lysine Methyl Transferases (KMTs) and Demethylases (KDMs), while acetylation is regulated by histone acetyltransferases (HATs) and histone deacetylases (HDACs). Lysine-specific Demethylase (LSD1 or KDM1A) demethylates histone H3K4me2/1 and, together with class I HDACs, works as subunits of repressive chromatin complexes such as Sin3, nucleosome remodeling and histone deacetylation (NuRD), corepressor for element-1-silencing transcription factor (CoREST), and nuclear receptor co-repressor/silencing mediator for retinoid or thyroid hormone receptors (NCoR/SMRT). Inhibition of either LSD1 or HDAC1 enzymes during early postnatal mouse retina development leads to a suppression of rod photoreceptor differentiation.

RP is an inherited form of retinal degeneration that is characterized by death of rod photoreceptors followed by secondary loss of cone photoreceptors. RP is very heterogeneous with over 4000 identified mutations in over 100 genes/loci. This is one reason for slow progress in identifying suitable treatments for patients. Several enzymes participating in the process of chromatin compaction and gene repression are upregulated in mouse models of RP.

Certain mouse mutations recapitulate many of the features of human retinitis pigmentosa. In the rd10 mouse line the rod-specific gene Pde6b is mutant; the same gene that is altered in one form of autosomal recessive retinitis pigmentosa in human. In these animals the mutation reduces but does not eliminate PDE6 activity and they display a phenotype where most retina cells reach terminal maturation before degeneration starts. Rapid degeneration happens between PN17 and PN25 with complete loss of rods observed by PN45-60.

Herein it is demonstrated that inhibition of LSD1 or HDAC1 in rd10 mice leads to rod photoreceptor preservation and maintenance of visual function. Analysis of the array of gene expression changes induced by these inhibitors indicates that they suppress expression of key inflammatory genes and also induce more open and accessible chromatin, which in turn allows expression of genes from a variety of neuroprotective mechanism. Manipulation of epigenetic modifiers represents a new strategy to fight neurodegeneration in RP.

### Methods

Antibodies and Reagents.

Chemicals were purchased from Fisher Scientific (Pittsburgh, PA), unless otherwise noted. 0.9% bacteriostatic sodium chloride was from APP Pharmaceuticals (Schaumburg, IL). LSD1 inhibitors: trans-2-Phenylcyclopropylamine (parnate or tranylcypromine, TCP) was purchased from Tocris Bioscience (Bristol, UK), GSK2879552 was from Selleckchem.com (Huston, TX). The HDAC inhibitor romidepsin was from Sigma (St. Louis, MO). Anti-rhodopsin (RHO) monoclonal antibodies have been described previously (Barnstable, 1980) and react with an N-terminal sequence shared by many species The commercial antibodies used were: anti-H3K4me2 (07-030, Upstate, Charlottesville, VA), anti-H3K9me2 (ab1220, Abcam, Cambridge, MA), anti-GFAP (MAB360, Millipore, Temecula, CA), anti-IBA1 (for the AO gene; 019-19741, Wako, Richmond, VA), anti-OPN1SW (AB5107, Millipore, Temecula, CA), anti-PRDE6B (PA1-722, Thermo Fisher Scientific, Wilmington, Delaware), anti-ACTB (A4700, Sigma, (St. Louis, MO).

Animals.

Wild type C57Bl/6J (cat #000664), and rd10 B6.CXB 1-Pde6brd10/Jrd10 (cat #004297) mice were purchased from Jackson laboratory (Bar Harbor, ME, United States) and housed in a room with an ambient temperature of 25 C, 30-70% humidity, a 12-h light-dark cycle, and ad libitum access to rodent chow. This study was carried out using both male and female mice in accordance with the National Research Council's Guide for the Care and Use of Laboratory Animals (8th edition) and all animal experiments were approved by the Pennsylvania State University College of Medicine Institutional Animal Care and Use Committee (protocol #46993).

Treatment with LSD1 and HDAC1 Inhibitors.

Mice were treated daily with intraperitoneal injections (i.p.) of trans-2-Phenylcyclopropylamine (parnate or tranylcypromine, TCP) at 10 mg/kg, GSK2879552 (GSK) at either 1.5 mg/kg or 4.2 mg/kg, romidepsin at 0.2 mg/kg, or saline as control. All inhibitors were diluted in 0.9% bacteriostatic sodium chloride (saline).

Tissue Collection.

Whole retinas were isolated from animals by removing the sclera and most of the retinal pigmented epithelium (RPE) layer under PBS. The right eye retinas from each animal were taken for RNA extraction, cDNA preparation and RT-PCR. Immediately after isolation, tissue was flash frozen in liquid nitrogen and stored at −80° C. Left eye retinas from each animal were subjected to fixation, cryopreservation, sectioning, and immunofluorescence staining.

RNA Extraction and cDNA Preparation.

RNA extraction and purification followed the manufacturer's protocol from RNeasy Mini Kit and RNA shredder (Qiagen). Final RNA concentrations were determined spectrophotometrically using a NanoDrop 1000 Spectrophotometer (Thermo Fisher Scientific, Wilmington, Delaware). cDNA was synthesized with SuperScript II, III or IV First-Strand Synthesis System kit according to manufacturer's protocol (Invitrogen, Carlsbad, California).

RT-PCR.

Primers purchased from Integrated DNA Technologies (IDT). The sequence information is listed in Table 1. For quantitative real-time PCR we used 2× iQ-SYBR Green PCR supermix from Bio-Rad. Samples in triplicate were run on an iQ5 Multicolor Real Time PCR Detection System (Bio-Rad). The relative expression level for each gene was calculated by the 2-ΔΔCt method and normalized to GAPDH. Genes were considered up or down-regulated if p value<0.05.

TABLE 1
Primer sequences to measure gene expression.

| | | Sequence | | Sequence |
|---|---|---|---|---|
| | | Identified | | Identified |
| Gene | Forward Primer | Numbers | Reverse Primer | Numbers |
| Aifm1 | TGCTCTTGGCAGAAAGTC | SEQ ID NO: 1 | TGGGCATCACTTTCAC | SEQ ID NO: 41 |
| | TC | | TCC | |
| Apaf1 | GTACACCCCCTGAAAAGC | SEQ ID NO: 2 | CAGGGTGGGTCACCAT | SEQ ID NO: 42 |
| | AA | | CTAT | |
| Bc12 | AGCCCGTGTTTGTAATGG | SEQ ID NO: 3 | CACAGCCTTGATTTTG | SEQ ID NO: 43 |
| | AG | | CTGA | |
| Bdnf | GAGCGTGTGTGACAGTAT | SEQ ID NO: 4 | CATGGGATTACACTTG | SEQ ID NO: 44 |
| | TAGCG | | GTCTCG | |
| C1qa | GAGGGGAGCCAGGAGCT | SEQ ID NO: 5 | GGATTGCCTTTCACGC | SEQ ID NO: 45 |
| | G | | CC | |
| C1qb | GCTGATGAAGACACAGTG | SEQ ID NO: 6 | GCTGTTGATGGTCCTC | SEQ ID NO: 46 |
| | GG | | AGG | |
| Capn2 | CCCCAGTTCATTATTGGA | SEQ ID NO: 7 | GCCAGGATTTCCTCAT | SEQ ID NO: 47 |
| | GG | | TCAA | |
| Casp9 | CAGGCCCGTGGACATTGG | SEQ ID NO: 8 | CAGCCGCTCCCGTTGA | SEQ ID NO: 48 |
| | TT | | AGATA | |
| Cend1 | Qiagen #QT00154595 | | | |
| Cd74 | CAAACCTGTGAGCCAGAT | SEQ ID NO: 9 | GGTCCTGGGTCATGTT | SEQ ID NO: 49 |
| | GC | | GC | |
| Cnga1 | AATACGTGGCATTCCTTC | SEQ ID NO: 10 | GAGCCATTGTCATCGT | SEQ ID NO: 50 |
| | GTAAA | | CAGAAA | |
| Cngb1 | AGAGGAGGAACACTACTG | SEQ ID NO: 11 | AAGTAATCCATGAGGA | SEQ ID NO: 51 |
| | CG | | GCCAGA | |
| Crx | Qiagen QT00115402 | | | |
| Cst7 | CGAACTACATGCAGGAAG | SEQ ID NO: 12 | CACTGGCAGAGGAGAA | SEQ ID NO: 52 |
| | ACC | | CAGG | |
| Cx3cr1 | CAGCATCGACCGGTACCT | SEQ ID NO: 13 | GCTGCACTGTCCGGTT | SEQ ID NO: 53 |
| | T | | GTT | |
| Foxn4 | GTGAGATCTACAGCTTCA | SEQ ID NO: 14 | TGAGATGAGCTTGTCC | SEQ ID NO: 54 |
| | TGAAGG | | AACTCC | |
| Foxp1 | GGTTGTACAGCAGTTAGA | SEQ ID NO: 15 | GGAGTATGAGGTAAGC | SEQ ID NO: 55 |
| | GCTACAG | | TCTGTGG | |
| Gapdh | Qiagen QT01658692 | | | |
| Gfap | GAGAGAAAGGTTGAATCG | SEQ ID NO: 16 | CGGCGATAGTCGTTAG | SEQ ID NO: 56 |
| | CTGG | | CTTC | |
| Gnat2 | ACCATGCCTCCTGAGTTG | SEQ ID NO: 17 | TGACTCTGGATCGAAG | SEQ ID NO: 57 |
| | | | CAC | |
| H2-Aa | GTCTTGACTAAGAGGTCA | SEQ ID NO: 18 | TTCTGAGCCATGTGAT | SEQ ID NO: 58 |
| | AATTCC | | GTTG | |
| Hes1 | TTCCAAGCTAGAGAAGGC | SEQ ID NO: 19 | GCACCTCGGTGTTAAC | SEQ ID NO: 59 |
| | AGAC | | GC | |
| Hes5 | AAGCTGCTGCTGGAGCAG | SEQ ID NO: 20 | GCAGCTTCATCTGCGT | SEQ ID NO: 60 |
| | | | GTC | |
| Neurod1 | CCTGTGACCTTTCCCATG | SEQ ID NO: 21 | AGAAGTGCTAAGGCAA | SEQ ID NO: 61 |
| | C | | CGC | |
| Nr2e3 | CTTCAAACCTGAAACACG | SEQ ID NO: 22 | CCTCAAAGATGGGAGC | SEQ ID NO: 62 |
| | AGG | | AGGAG | |
| Nrl | GTGCCTCCTTCACCCACC | SEQ ID NO: 23 | GCGTGCGGCGCCTCTG | SEQ ID NO: 63 |
| | TTCAGTGA | | CTTCAGCCG | |
| Opn1sw | CAGCATCCGCTTCAACTC | SEQ ID NO: 24 | GCAGATGAGGGAAAGA | SEQ ID NO: 64 |
| | CAA | | GGAATGA | |
| Otx2 | TCGCCACCTCTACTTTGA | SEQ ID NO: 25 | AGCCGCATTGGACGTT | SEQ ID NO: 65 |
| | TAG | | AG | |
| Pde6b | CTGACGAGTATGAGGCCA | SEQ ID NO: 26 | TAGGCAGAGTCCGTAT | SEQ ID NO: 66 |
| | AAG | | GCAGT | |
| Pde6c | CCTTATGTGGTCAGCCAA | SEQ ID NO: 27 | CCATCTGGAGTCTTTG | SEQ ID NO: 67 |
| | TAAAG | | GTCC | |
| Prph2 | TGGATCAGCAATCGCTAC | SEQ ID NO: 28 | CTGTAGTAATTCAGCA | SEQ ID NO: 68 |
| | CT | | GAGC | |
| Ptp4a3 | TACAGAGCTTCCTCCAAG | SEQ ID NO: 29 | CACGGTGTTGGGAACG | SEQ ID NO: 69 |
| | GAAA | | G | |
| Ptprc | TGCCTCACCTACACACAC | SEQ ID NO: 30 | ACATGAGTCATTAGAC | SEQ ID NO: 70 |
| | C | | ACACTGATG | |
| Rgr | TTGTGTGGATGTCATCTG | SEQ ID NO: 31 | GAAGTGTGTGTGATGA | SEQ ID NO: 71 |
| | C | | ACAGG | |
| Rho | CTTCTCCAACGTCACAGG | SEQ ID NO: 32 | GGACCACAGGGCGATT | SEQ ID NO: 72 |
| | CGT | | TCAC | |
| Rlbp1 | AGCAGGGCTTTGATGGTA | SEQ ID NO: 33 | TTCAGCTCATCCTTGG | SEQ ID NO: 73 |
| | GC | | CCTTC | |
| Rom1 | Qiagen #QT00172165 | | | |
| Rorb | CCGTCAGAATGTGTGAGA | SEQ ID NO: 34 | ATCCTCCCGAACTTTA | SEQ ID NO: 74 |
| | ACCAG | | CAGCATC | |
| Sag | GCCATGAGTGTCCTCACC | SEQ ID NO: 35 | GGCATGCTGCACTTTC | SEQ ID NO: 75 |
| | | | C | |
| Samd11 | CTTTCTGGCTGTGGCGAG | SEQ ID NO: 36 | GCCATGTAGAAGACAC | SEQ ID NO: 76 |
| | | | GGC | |
| Smad3 | GTGAAGAAGCTCAAGAAG | SEQ ID NO: 37 | ACTGGAGGTAGAACTG | SEQ ID NO: 77 |
| | AC | | GCGTC | |
| Sox2 | GAGTGGAAACTTTTGTCC | SEQ ID NO: 38 | GAAGCGTGTACTTATC | SEQ ID NO: 78 |
| | GAG | | CTTCTTCAT | |
| Tfap2b | Qiagen #QT00135478 | | | |
| Thrb | GTTTTCCCTCTCGTCCAT | SEQ ID NO: 39 | GCTTCCGCTTGGCTAG | SEQ ID NO: 79 |
| | CAGAGGACCTG | | CCTCTTGCT | |
| Vsx2 | ACGGAGCTCCCAGAAGAC | SEQ ID NO: 40 | CCATCCTTGGCAGACT | SEQ ID NO: 80 |
| | | | TG | |

RNA-seq.

RNA was extracted from PN24 retina of WT and rd10 animals treated with saline or GSK from PN9 till PN24. RNA integrity number (RIN) was measured using BioAnalyzer (Agilent Technologies) RNA 6000 Nano Kit to confirm RIN above 7 for each sample. The cDNA libraries were prepared using the Illumina® Stranded mRNA Prep, Ligation kit (Illumina) as per the manufacturer's instructions. Briefly, polyA RNA was purified from 200 ng of total RNA using oligo (dT) beads. The extracted mRNA fraction was subjected to fragmentation, reverse transcription, end repair, 3′— end adenylation, and adaptor ligation, followed by PCR amplification and SPRI bead purification (Beckman Coulter). The unique dual index sequences (IDT® for Illumina® RNA UD Indexes Set A, Ligation, Illumina) were incorporated in the adaptors for multiplexed high-throughput sequencing. The final product was assessed for its size distribution and concentration using BioAnalyzer High Sensitivity DNA Kit (Agilent Technologies). The libraries were pooled and diluted to 3 nM using 10 mM Tris-HCl, pH 8.5, and then denatured using the Illumina protocol. The denatured libraries were loaded onto an S1 flow cell on an Illumina NovaSeq 6000 (Illumina) and run for 2×53 cycles according to the manufacturer's instructions. De-multiplexed and adapter-trimmed sequencing reads were generated using Illumina bcl2fastq (released version 2.20.0) allowing no mismatches in the index read. BBDuk was used to trim/filter low-quality sequences using “qtrim=lr trimq=10 maq=10” option. Next, alignment of the filtered reads to the mouse reference genome (mouse Ensembl release 67 (GRCm37/NCBIM37/mm9)) was done using HISAT2 (version 2.1.0) applying --no-mixed and --no-discordant options. Read counts were calculated using HTSeq by supplementing Ensembl gene annotation (release 67: “Mus_musculus.Ensembl.NCBIM37.67.gtf”). The edgeR R package was used to fit the read counts to the negative binomial model along with the generalized linear model (GLM) and differentially expressed genes were determined by the likelihood ratio test method implemented in the edgeR. Significance was defined to be those with q-value<0.05 calculated by the Benjamini-Hochberg method to control the false discovery rate (FDR) and log 2 fold change is greater than 1 or smaller than −1. The ggplot2 R package was used for generating heatmaps. Raw counts and differential expression analysis generated during this study are available at GEO Submission GSE169527.

Pathway, Gene Ontology and Upstream Regulator Analysis.

Ingenuity Pathways Analysis (IPA) was used to identify upstream regulators and significantly enriched canonical pathways with following cutoff of FDR<0.05 and fold change in gene expression bigger than 2 or smaller than ½. The Database for Annotation, Visualization, and Integrated Discovery (DAVID) was used for GO functional analysis.

Immunofluorescence Staining.

Retinas were fixed in 4% paraformaldehyde overnight at 4° C., washed in PBS, incubated in 5% sucrose/PBS for 30 min and then cryopreserved in 20% sucrose/PBS overnight at 4° C. Retinas were embedded in 2:1 mix of 20% sucrose and OCT (Sakura Finetek Torrance, CA) and stored at −80° C. Blocks with tissue samples were sectioned to 7-10 μm on a Cryostat Microtome HM550 (Thermo Fisher Scientific) and stored at −20° C. Antigen retrieval was performed by incubating the slides in 10 mM sodium citrate pH 6 for 30 min at 80° C. Double labeling immunohistochemistry was performed using fluorescent Alexa Fluor-conjugated secondary antibodies diluted 1:800 (Invitrogen, Carlsbad, California). Primary antibodies were diluted as follow: anti-H3K4me2 1:600, anti-H3K9me2 1:600, anti-RHO 1:50, anti-OPN1SW 1:400, anti-GFAP 1:1000, anti-IBA1 1:450 (AifI gene). Slides were counterstained with Hoescht 33258 (1 mg/ml diluted 1:1000) and visualized using an Olympus Fluoview FV1000 confocal microscope (Olympus Center Valley, PA). The acquisition parameters were maintained constant for each set of experiments. Fluorescence intensity was assessed using ImageJ software (Bethesda, MD).

Western Blot.

Both retinas from one animal were flash frozen together in liquid nitrogen and stored at −80° C. Samples for SDS-PAGE were prepared by resuspended retinas in 50 μl PBS. Laemmli sample buffer was added, samples were boiled for 10 min, resolved on Criterion TGX Precast Gel AnyKD (Bio-Rad), transferred to nitrocellulose membrane, and immunoblotted with antibodies anti-H3K9me2 or anti-H3K4me2 diluted 1:5000; anti-ACTB diluted 1:10000; anti-PDE6B (1:500). Secondary goat anti-rabbit/mouse HRP (Jackson Immuno-Research Laboratory, West Grove, PA) was diluted 1:5000. An ECL western blot detection system (Thermo Fisher Scientific) was used to visualize the bound of the primary antibody.

Chromatin Immunoprecipitation (ChIP).

Lysate preparation for ChIP was carried out. In brief, 10 mouse retinas were rapidly isolated and rinsed in PBS on ice. Cell suspensions in PBS were crosslinked with 1% formaldehyde for 15 min at room temperature, followed by quenching with 1 M glycine, incubation on ice for 5 min, and centrifugation for 7 min at 4,000 rpm at 4° C. Pellets were resuspended in 500 μl L-CHIP buffer (1% SDS, 10 mM EDTA, 50 mM Tris-HCl pH 8.0), 1 mM PMSF and PI, sonicated twice at setting 3 for 10 sec on Sonic Dismembrator (Fisher Scientific, Model 100). Protein concentrations were adjusted to 1 mg/ml with L-CHIP buffer. ChIP was performed and subjected to quantitative PCR, primers for genomic regions are specified in Table 2.

TABLE 2
Primer sequences for ChIP assay

| | | Sequence | | Sequence | |
|---|---|---|---|---|---|
| | | Identifier | | Identifier | Target |
| Gene | Forward Primer | Number | Reverse Primer | Number | Region |
| Bdnf | AGCTGCGGGTATCTCCA | SEQ ID | AGGCTGAGATCCTAGGC | SEQ ID | Promoter |
| | TAA | NO: 81 | AGA | NO: 95 | |
| Cend1 | TTAGAATAAAGCGGTTC | SEQ ID | TTCGGAGCTACAGTGGA | SEQ ID | Promoter |
| | CACC | NO: 82 | ATC | NO: 96 | |
| Crx | GCTCAGGTTGGCCTCAG | SEQ ID | CCACACTAGTGCGAGAC | SEQ ID | Promoter |
| | AC | NO: 83 | CTGAG | NO: 97 | |
| Foxn4 | GGACTTTGATCCTGCTG | SEQ ID | CAAGGGTTCTAGGAAAT | SEQ ID | Intragenic, |
| | CAAG | NO: 84 | GGTCC | NO: 98 | H3K4me2 |
| | | | | | max in |
| | | | | | embrYo |
| Foxp1 | AGATAGAAGGTGCAGCA | SEQ ID | AGTTGTAACTCCAAACC | SEQ ID | Promoter |
| | AGAAGG | NO: 85 | TTGCG | NO: 99 | |
| Gnat2 | GAACAGAGACTGCAGAG | SEQ ID | AATCTTGATGGTGTCTG | SEQ ID | Intragenic, |
| | ACAGATC | NO: 86 | ATATTGG | NO: 100 | CRX, NRL |
| | | | | | binding |
| Hes1 | GCCTGGCCACAAAAGAA | SEQ ID | CCCAAACTTTCTTTGCC | SEQ ID | Proximal |
| | ATA | NO: 87 | ACA | NO: 101 | promoter |
| Otx2 | CACGGTCACTTCTCCAC | SEQ ID | CCCCATTCCCCTTTGCA | SEQ ID | Promoter |
| | AGCG | NO: 88 | CTG | NO: 102 | |
| Pde6b | CAGGACCCGTTTCATCA | SEQ ID | GGTGTTCCTGCTGCTGT | SEQ ID | Promoter |
| | G | NO: 89 | G | NO: 103 | |
| Rgr | CAGGCAAGCTACAGACC | SEQ ID | TAATATGCAGGAATCCT | SEQ ID | Enhancer in |
| | TCAG | NO: 90 | TCATGG | NO: 104 | the end of |
| | | | | | gene |
| Rho | GGAATTCCCAGAGGACT | SEQ ID | CTCTTCGTAGACAGAGA | SEQ ID | Promoter |
| | CTG | NO: 91 | CC | NO: 105 | |
| Smad3 | TGAAGCCCACACAGGAA | SEQ ID | CTATAAGCATCCATATG | SEQ ID | Intragenic |
| | GTG | NO: 92 | CACTGTGG | NO: 106 | |
| Sox2 | AAAGCACTCTAGTAAAA | SEQ ID | GGTGTACTATTACGCTC | SEQ ID | Enhancer |
| | GCAAGTCC | NO: 93 | AACACG | NO: 107 | end of loop |
| Vsx2 | TTCTGCTGCTGTTCCTC | SEQ ID | CTAATAACGATGGTATT | SEQ ID | Proximal |
| | ATTTAC | NO: 94 | GGCTCAG | NO: 108 | promoter |

Visual Function.

A Cerebral Mechanics OptoMotry system (CerebralMechanics Inc.) was used to evaluate visual function in control and GSK ESD rd10 mice. Spatial frequency (SF) threshold and contrast sensitivity (CS) were assessed using a video camera to monitor optomotor reflex. CS was assessed at a SF of 0.092 cycles/degree. SF was assessed at 100% contrast. The CS and SF thresholds were identified as the highest values that elicited the reflexive head movement. SF (acuity) was measured on PN24, PN26, PN28 and PN32 and then averaged, during this time acuity in GSK treated animals slowly increased, while control mice acuity was diminished. CS were measured three times on PN33-35, when control mice were blind.

TUNEL Assay.

Retinas were fixed in 4% paraformaldehyde overnight at 4° C., washed in PBS, incubated in 5% sucrose/PBS for 30 min and then cryopreserved in 20% sucrose/PBS overnight at 4° C. Retinas were embedded in 2:1 mix of 20% sucrose and OCT (Sakura Finetek Torrance, CA) and stored at −80° C. Blocks with tissue samples were sectioned to 7-10 μm on a Cryostat Microtome HM550 (Thermo Fisher Scientific) and stored at −20° C. TUNEL assay was carried out using the In situ Cell death detection kit, Fluorescein from Roche (Germany) according to manufacturer's instruction. Sections were counterstaining with Hoescht 33258 (1 mg/ml diluted 1:1000), washed 3 times with PBS and analyzed by confocal microscopy.

Statistical Analyses.

Results are presented as means±standard error of the mean (SEM). Unpaired, one tail Student's t-test (two-tailed, unpaired) was used to evaluate statistical significance between groups. P value<0.05 was considered significant. Statistical analyses for experiments were performed using the GraphPad Prism software.

Results

LSD1 and HDAC1 Inhibitors Preserve Rod Photoreceptors in Mouse Models of Retinitis Pigmentosa.

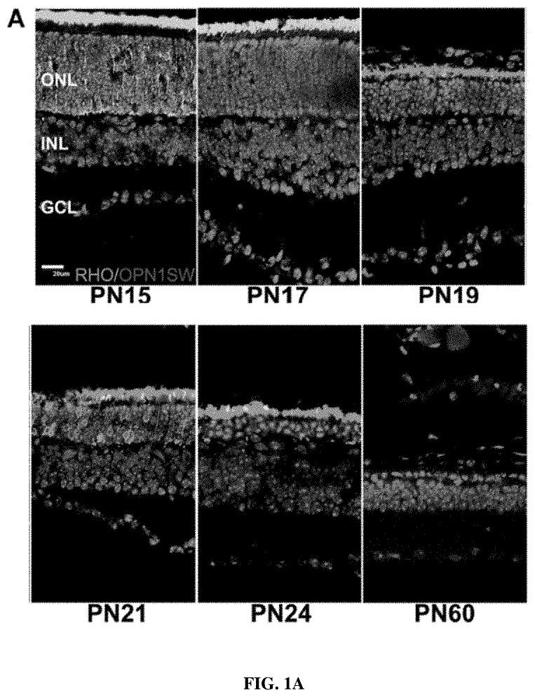

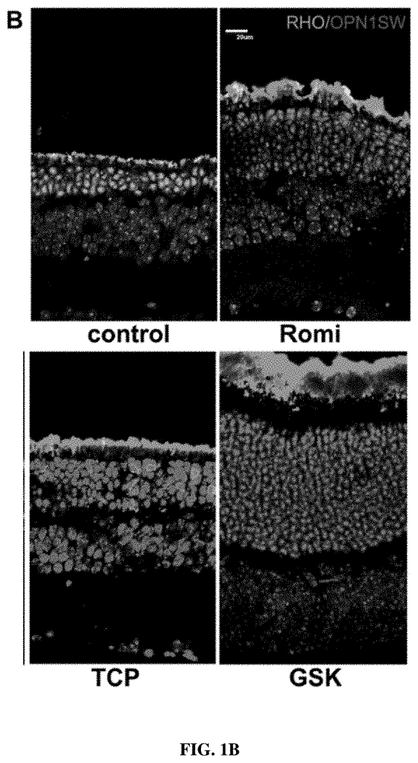

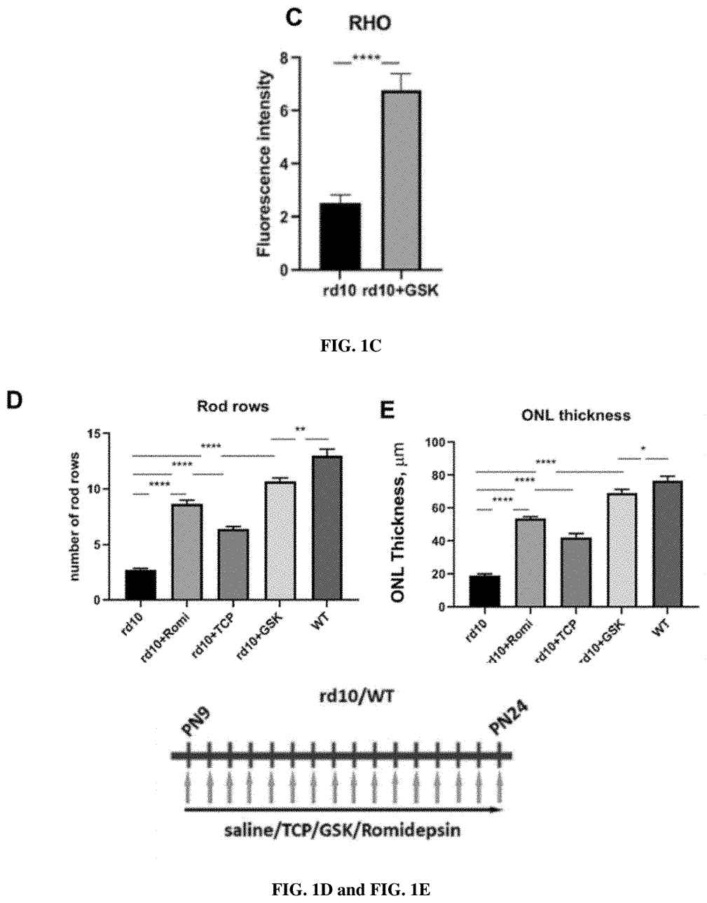

It has been shown that LSD1 and HDAC1 inhibitors have dramatic effects on the expression of rod photoreceptor genes during retinal development. Herein, a mouse model of retinitis pigmentosa is being tested to determine whether the changes induced by these inhibitors could also offset the degenerative changes found in this disease. The phenotype of rd10 is illustrated in FIG. 1A where loss of rods is apparent by PN19, most photoreceptors have disappeared by PN24, and photoreceptor loss is complete by PN60.

The rd10 mice were treated daily with intraperitoneal injection (i.p.) of the HDAC1 inhibitor romidepsin, the LSD1 inhibitors tranylcypromine (TCP) and GSK2879552 (GSK), or control saline for 15 days beginning at PN9, several days before degeneration is detectable, and ending at PN24, when most rod photoreceptors have normally been lost.

In control rd10 mice injected with saline only 2-3 rows of photoreceptors remained in the outer nuclear layer (ONL) of the retina. Based on antibody staining, most of these were rods, although the lengths of their outer segments (OS) were diminished (FIG. 1B). Treatment of rd10 mice with romidepsin at a concentration of 2 mg/kg was harmful as mice were dying during such treatment. Lower concentrations of romidepsin also increased mortality but a concentration of 0.2 mg/kg was used, where the effect of the treatment was seen even though mice were not healthy (discussed later). Under romidepsin inhibition an average of 8 rows of photoreceptor remained in the ONL at PN24, but OS were not so pronounced as after TCP treatment (FIGS. 1B and 1D). Treatment with TCP at a concentration of 10 mg/kg partly protected ONL with in average 7 rows of photoreceptors remaining at PN24 (FIGS. 1B and 1D) with longer OS. The best retina ONL preservation was achieved when we treated rd10 mice with the more specific LSD1 inhibitor GSK. The starting concentration for mice was 1.5 mg/kg, but the effect on photoreceptor preservation was mild (5-6 rows of photoreceptors remain; data not shown), so the dose was increased to 4.2 mg/kg and all subsequent experiments were done with this concentration. Treatment with this concentration of GSK led to preservation of more than 10 rows of photoreceptors (FIGS. 1B and 1D), almost to the level of wild type (WT) retina ONL (FIGS. 1D and 1E) with normal length OS. RHO protein staining was also increased under GSK inhibition (FIG. 1C). In addition to counting photoreceptor rows in ONL, we measured the overall ONL thickness (FIG. 1E). The patterns of changes in ONL thickness closely followed the changes in the number of photoreceptors rows in ONL.

Continuous Presence of LSD1 Inhibitors is Needed to Prevent Rod Degeneration in a Retinitis Pigmentosa Model.

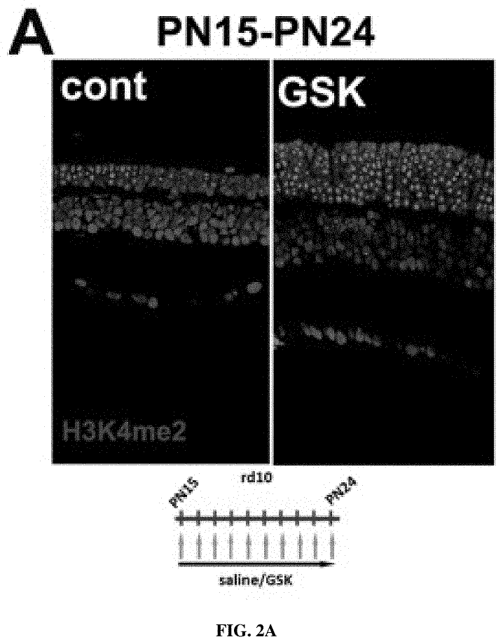

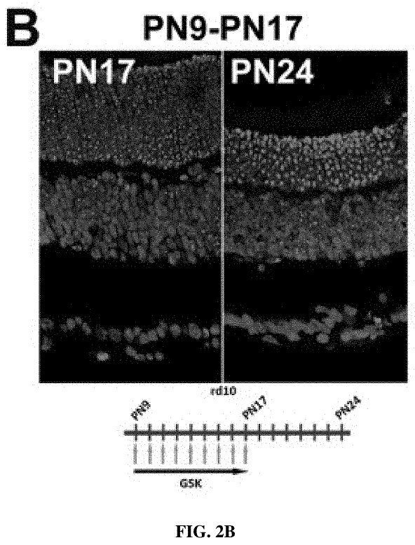

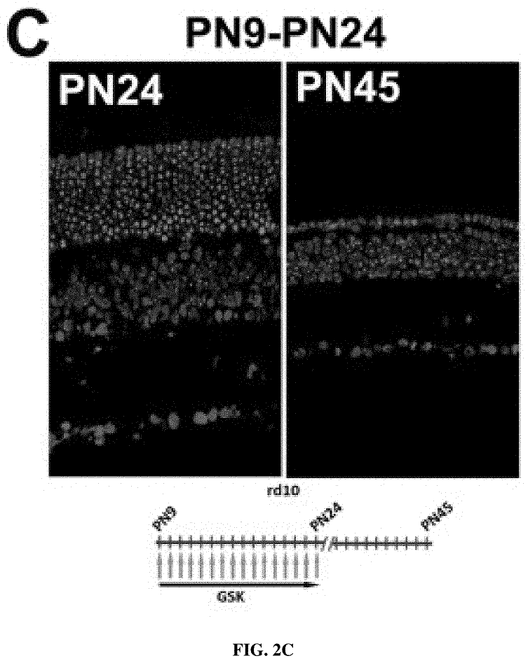

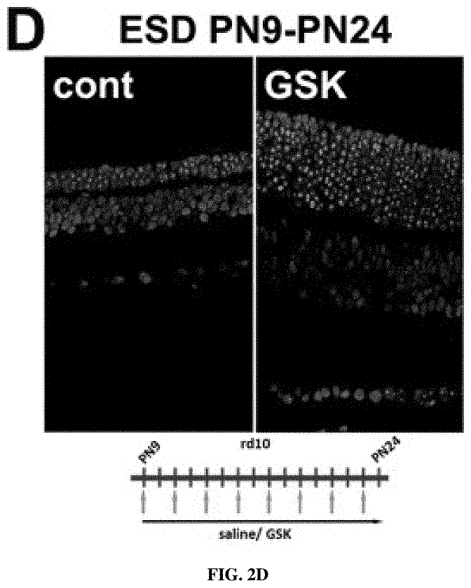

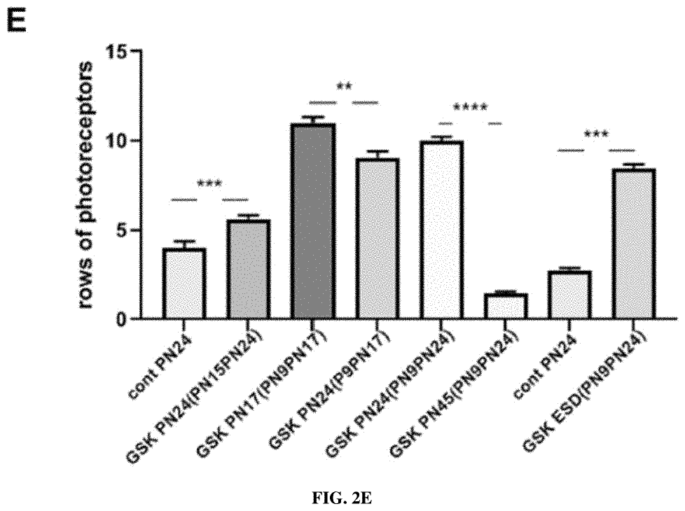

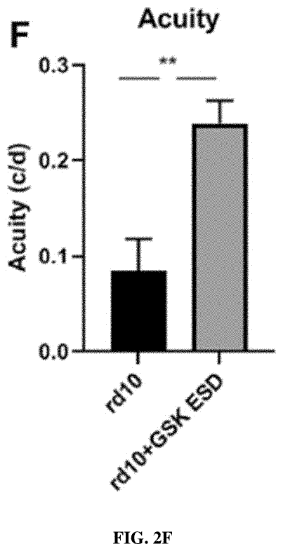

Next, a series of experiments was carried out to investigate the time course of the effects of GSK treatment. First, the result of beginning the injection of GSK at a later time point was examined. Treating mice with GSK from PN15 to PN24 blocked further degeneration (FIG. 2A, E, pink bars). Treatment from PN9 to PN17 showed minimal degeneration when the retinas were examined at PN17, but substantial loss of photoreceptors at PN24, one week after ending treatment (FIG. 2B, E, gray bars). Similarly, when mice were treated with the original time course, from PN9 to PN24, the preservation of rod photoreceptors seen at PN24 decreases over the next 21 days so that at PN45 there had been substantial loss of cells (FIGS. 2C and 2E, yellow bars). These results argue that GSK can block but not reverse degeneration and that its continued presence is necessary to prevent further rod photoreceptor degeneration in the rd10 mutant.

Next, it was also tested whether daily doses of GSK were necessary. Mice were treated each second day (ESD) with GSK 4.2 mg/kg for the same length of time as before and in these conditions, photoreceptors have survived to essentially the same extent as with treatment each day (FIGS. 2D and 2E, green bars).

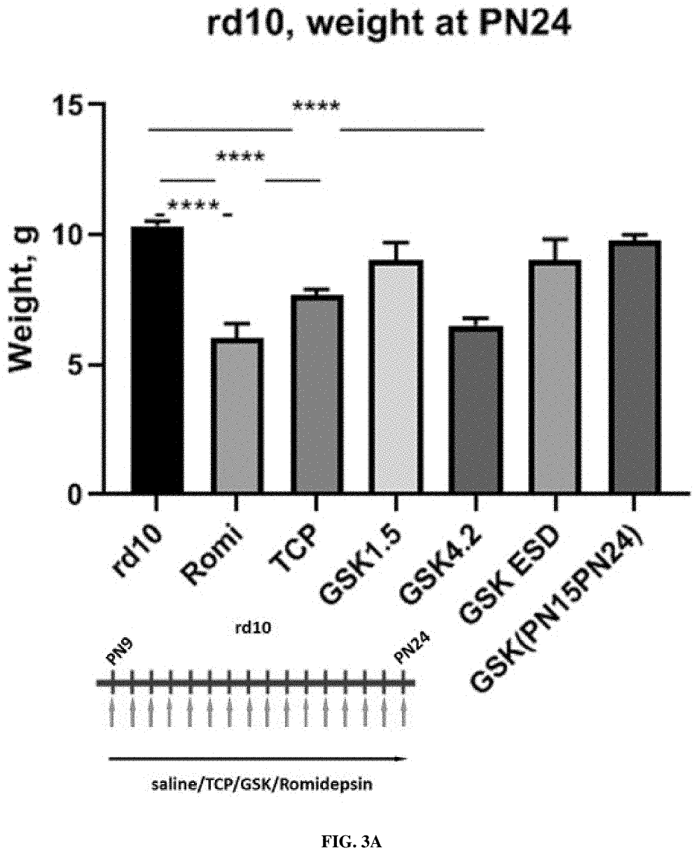

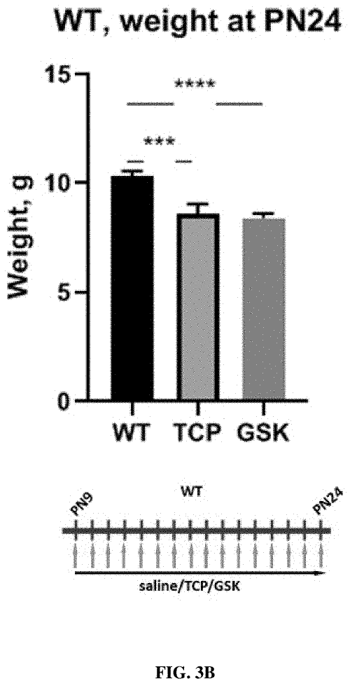

During all treatments animal weight gain was monitored. All mice treated with inhibitors, both mutant and WT, showed less weight gain when compared with saline injected controls (FIGS. 3A and 3B). The smallest increase in body weight was observed with romidepsin treatment. GSK in smaller concentration 1.5 mg/kg or injecting higher 4.2 mg/kg each second day (ESD) did not show any significant difference in body weight gain from controls.

Treatment of Rd10 Mice with an LSD1 Inhibitor Increases Visual Function.

As ESD treated mice had better movement and reflexes visual functions were tested in this group of mice using optometry reflex. After treatment with GSK, rd10 mice demonstrated much better acuity than saline treated controls (FIG. 2F), where untreated mutant animals have spatial frequency threshold around only 0.085 cyc/deg, while GSK injected mice have threshold around 0.240 cyc/deg. Acuity in WT mice reached a maximum of 0.4 cyc/deg, so the treatment preserves around 60% of vision in rd10 mice. Spatial frequency was measured on PN24, PN25, PN26, PN28 and PN32, and during this time frame acuity in GSK treated animals slowly increased, while in untreated mice acuity was diminished (data not shown). Additionally, GSK treated mice had contrast sensitivity 43.4+/−14.6% at 0.092 c/d spatial frequency when measured at PN33-35. Maximum contrast sensitivity in WT mice reached 95% at this spatial frequency, so the treatment preserved approximately 50%. At this age, the control rd10 mice were blind. Thus, inhibition of LSD1 in mouse models of Retinitis Pigmentosa (RP) not only leads to morphological rod photoreceptor preservation, but also helps to maintain visual function.

Inhibitor Specific for LSD1 Alters Retinal Gene Expression in Rd10 Mice.

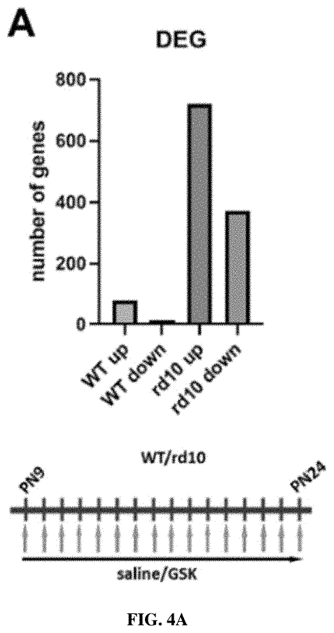

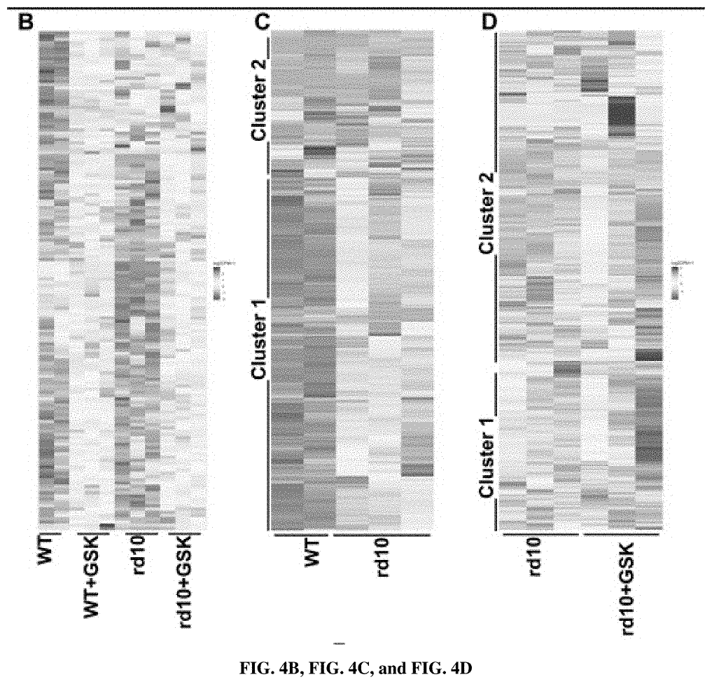

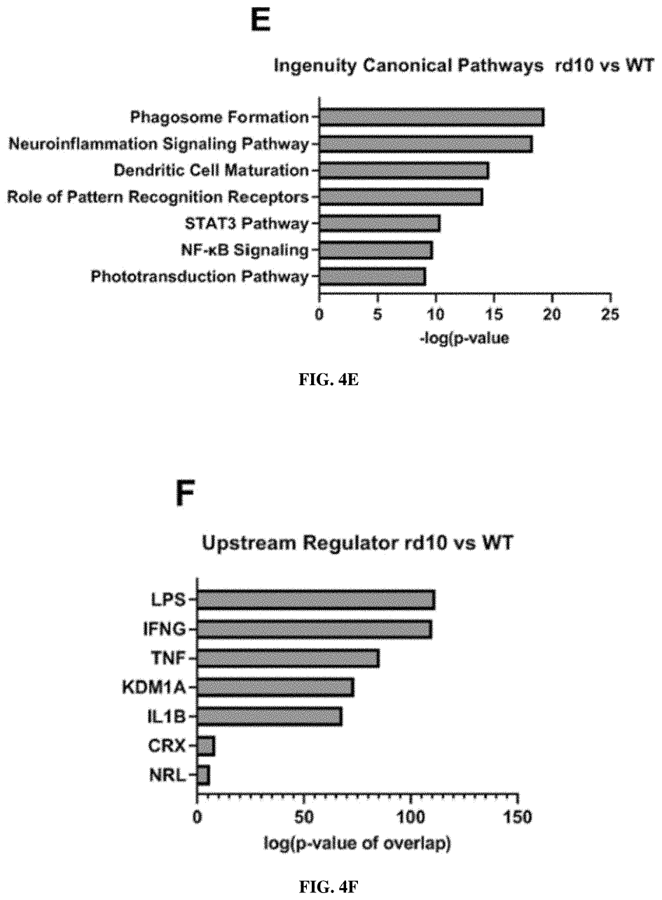

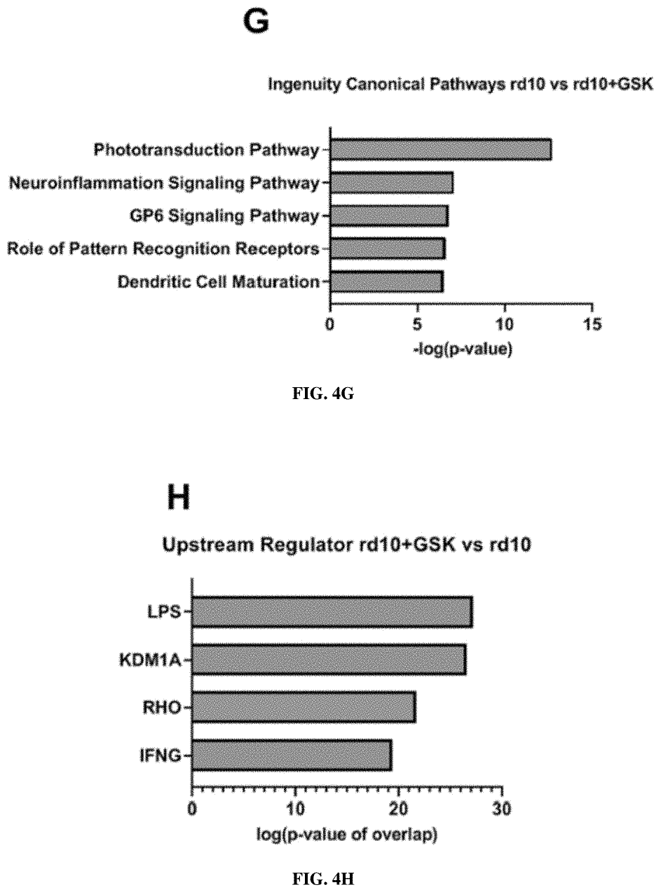

After establishing that treatment of mouse models of Retinitis Pigmentosa with inhibitors specific for LSD1 and HDAC1 led to morphological and functional preservation of rod photoreceptors it was next studied how this treatment influences gene expression. RNA-seq was performed on retina samples from rd10 and WT mice treated with GSK inhibitor or saline from PN9 till PN24 (FIG. 4). Using a cutoff of FRD<0.05 and a fold change greater than 2 or smaller than 0.5, rd10 mice treated with GSK have 719 genes upregulated and 369 genes downregulated, while in WT mice GSK only upregulated 77 genes and downregulated 13 genes (FIG. 4A).

With more relaxed criteria (p<0.05 and a fold change bigger than 1.75 or smaller than 0.8) approximately 150 genes were found that were simultaneously upregulated in rd10 and WT mice retina under GSK inhibition (FIG. 4B). It was contemplated that these differentially expressed genes (DEG) found in both rd10 and WT are upregulated because of global effects on chromatin structure and accessibility. To test this the chromatin state was examined, using databases for adult WT mice retina, of the 147 genes that have annotation in RefSeq database, 39 DEG are in heterochromatin, 8 are on the border between euchromatin and heterochromatin and 8 are in euchromatin but show decreasing expression during development. 53 DEG have no histone epigenetic marks, H3K27 or H3K4me2, and 57 DEG have more inhibitory marks (H3K27me3), than active marks (H3K4me2). These findings demonstrate that the majority of the DEG simultaneously upregulated by GSK in rd10 and WT mice belong to normally repressed or silent chromatin compartments in adult retina and their upregulation under LSD1 inhibition indicates an opening of chromatin. The H3K4me2 histone modification is a marker not only of transcribed genes, but also of enhancers and 38 DEG genes that were in euchromatin are in a vicinity of developmental superenhancers (+/50 kb). Among upregulated common genes were developmental transcriptional factors (Cited4, Foxf1, Gsc2, Irf6, Lefty2, Mesp1, Mesp2, Myod1, Pax7, Sox10, Zcchc12) and genes participating in eye development and homeostasis (Arhgap36, Baiap3, Ccno, Cuta1, Crabp2, Gng8, Lrat, Mapk15, Sfrp5, Sypl2). Several genes belong to the WNT pathway and could play an important role in neuroprotection (Fam83g, Lefty2, Sfrp5, Upk1b).

Next genes and pathways that were differentially expressed in rd10 in comparison to WT retina were analyzed (FIG. 4C). Two clusters readily classified the DEG. In cluster 1 genes were upregulated in rd10 and in cluster 2 genes were down-regulated in rd10. Ingenuity Pathways Analysis (IPA) demonstrated that pathways associated mostly with inflammation and phagocytosis were activated (cluster 1), while the phototransduction pathway was inhibited (cluster 2) (FIG. 4E). Upstream regulators for activated pathways, according to IPA, were LPS, IFNG, TNF, IL6B, all connected to inflammation; upstream regulators for inhibited pathways were CRX and NRL (FIG. 4F).

We then identified DEG in rd10 retina under LSD1 inhibitor (FIG. 4D). Again, all genes sorted into two clusters, where cluster 1 comprises genes that are down regulated in rd10+GSK retinas and in cluster 2 consist of genes that are up-regulated in rd10+GSK. IPA indicated that the major upregulated pathway is the phototransduction pathway (FIG. 4G). The next 4 most important down regulated pathways were all related to neuroinflammation (FIG. 4G). According to IPA, top upstream regulators were LPS (lipopolysaccharide) and IFNG (Interferon), connected to inflammation pathways, and RHO (rhodopsin), and connected to the phototransduction pathway (FIG. 4H). The key finding was that KDM1a (another name for LSD1) is the second most important upstream regulator. This supports the recent finding that LSD1 can act on non-histone targets to regulate the inflammatory response.

The RNA-seq data was then extended with a GO analysis of the functions of DEG in rd10 compared to WT retina (Table 3) and in rd10+GSK compared to rd10 (Table 4). The top biological processes that were upregulated in rd10 and reverted back by GSK treatment are innate immune response and immune system process, most crucial cellular components were membrane, external side of plasma membrane, and extracellular region; molecular functions were 2′-5′ oligoadenylatase activity, peptide antigen binding and cytokine receptor activity. The most essential biological processes that were downregulated in rd10 and reverted back by GSK were visual perception, response to stimulus, and phototransduction; the top cellular components were photoreceptor outer and inner segments and extracellular matrix; the top molecular functions were structural constituents of eye, and cGMP binding.

TABLE 3
Functional enrichment for gene clusters in FIG. 4C identified
using a web-based DAVID classification tool and GO categories.

| Cluster | WT versus rd10 | Biological process | Cellular component | Molecular function |
|---|---|---|---|---|
| Cluster 1 | UP in rd10 | Immune system | Extracellular region | Integrin binding |
| | | process | | |
| | | Innate immune | Cell surface | Heparin binding |
| | | response | | |
| | | apoptotic process | Phagocytic vesicle | Peptide antigen |
| | | | membrane | binding |
| | | Phagocytosis | External side of | TNF-activated |
| | | | plasma membrane | receptor activity |
| | | Positive regulation | Pernuclear region | 29-59- |
| | | of inflammatory | of cytoplasm | oligoadenylate |
| | | response | | synthetase activity |
| Cluster 2 | DOWN in rd10 | Visual perception | Photoreceptor outer | Structural |
| | | | segment | constituent of eye |
| | | Response to | Photoreceptor inner | Intracellular |
| | | stimulus | segment | cGMP-activated |
| | | | | cation channel |
| | | | | activity |
| | | Phototransduction | Cell projection | Haptoglobin |
| | | | | binding |
| | | Cilium assembly | Extracellular matrix | Transporter |
| | | | | activity |
| | | Neurotrasmitter | | |
| | | transport | | |

TABLE 4
Functional enrichment for gene clusters in FIG. 4D identified
using a web-based DAVID classification tool and GO categories.

| Cluster | rd10 vs rd101GSK | Biological process | Cellular component | Molecular function |
|---|---|---|---|---|
| Cluster 1 | DOWN in rd101GSK | Immune system | Membrane | Peptide antigen |
| | | process | | binding |
| | | Innate immune | External side of | 29-59-oligoadenylate |
| | | response | membrane | synthetase activity |
| | | Pyroptosis, | Extracellular | Beta-2-microglobulin |
| | | cytolysis | exosome | binding |
| | | Phagocytosis | Cell surface | Cytokine receptor |
| | | engulfment | | activity |
| | | Interleukin-1 beta | NLPR1 | Carbohydrate binding |
| | | production | inflammasome | |
| | | | complex | |
| Cluster 2 | UP in rd101GSK | Visual perception | Photoreceptor outer | Structural constituent |
| | | | segment | of eye |
| | | Response to | Extracellular | Bicarbonate |
| | | stimulus | matrix | transmembrane |
| | | | | transport activity |
| | | Positive regulation | Photoreceptor inner | Protein |
| | | of rhodopsin gene | segment | heterodimerization |
| | | expression | | activity |
| | | Phototransduction | Extracellular region | cGMP binding |
| | | Retinol metabolic | Z disk | |
| | | process | | |

Next, the type of immune cells participating in the inflammation process was explored in rd10 retinas and what type of immune cells were the major targets for GSK inhibition. Known cell markers for Muller glia, such as Rlbp1, Slc1a3, Glast, Ptbp1, Cralbp1, Glu1 were not changed in rd10 vs WT or in rd10 vs rd10+GSK according to RNA-seq data. However, some Muller cell markers of damaged retinas, including Lcn2, Serpina3e, Gfap, Cxcl10, Timp1, Ccl2 were up-regulated in rd10 in comparison with WT, but not all of them returned back to a WT level of expression in rd10+GSK retina. On the other hand, many more markers for activated microglia and infiltrating immune cells were up-regulated in rd10 retinas (markers according to. Expression of resident microglia markers Atp6v0d2, Mcoln3, Cd5l, Ctsd, C1qa, Fcrls, Hexb, Gpr34, Junb, Pclaf, Birc5 were upregulated in rd10 and went back to normal levels of expression in rd10+GSK. Expression of markers of infiltrating immune cells, such as monocytes and macrophages, H2-Aa, cd74, H2-Ab1, H2-Eb1, Cyp4f18, Ms4a6c, Lyz2, Ms4a7, 4930430Erik, Apoe, Hp, Ly6c2, Cx3cr1 were also upregulated in rd10 and returned to normal levels of expression in rd10+GSK. This shows that the major inflammatory mediators in rd10 were associated with microglia and that these were susceptible to inhibition by LSD1 inhibitors.

Validation of RNA-Seq Results by qPCR.

Selected results of the RNA-seq experiments were confirmed using qRT-PCR where genes were considered up- or down-regulated if the expression was significantly different from untreated control rd10 animals with a p value<0.05. Allowing for the different thresholds and sensitivities of the methods, the qPCR results verify the conclusions of the RNA-seq study. Each of the classes of genes validated by qPCR are described and illustrated in the following subsections.

Changes in Rod Photoreceptor Gene Expression.

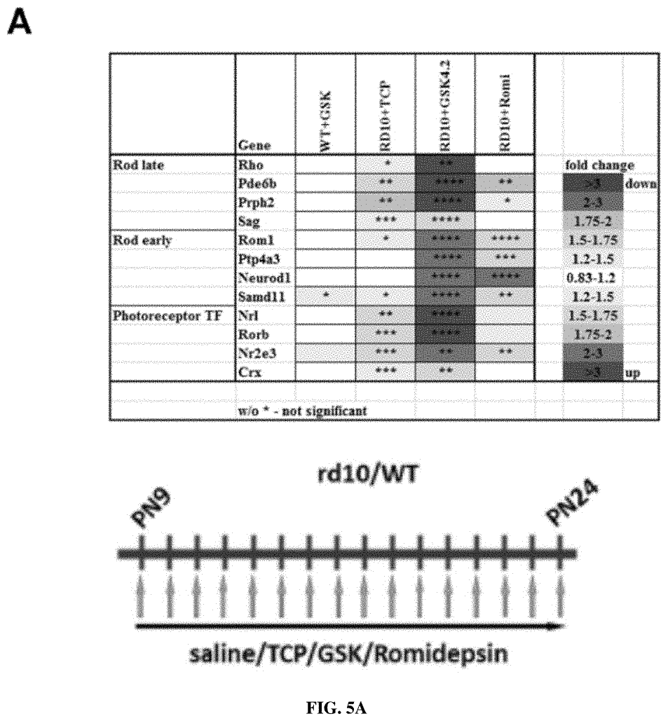

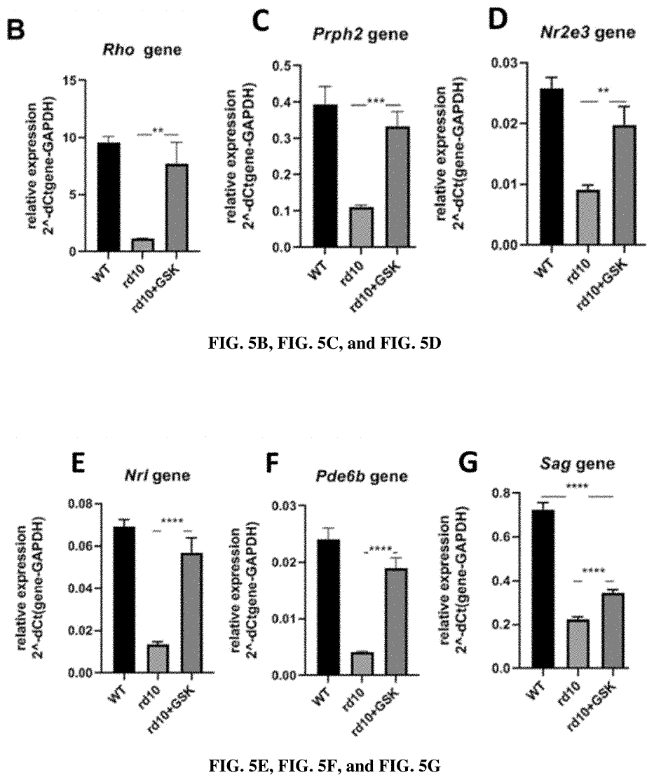

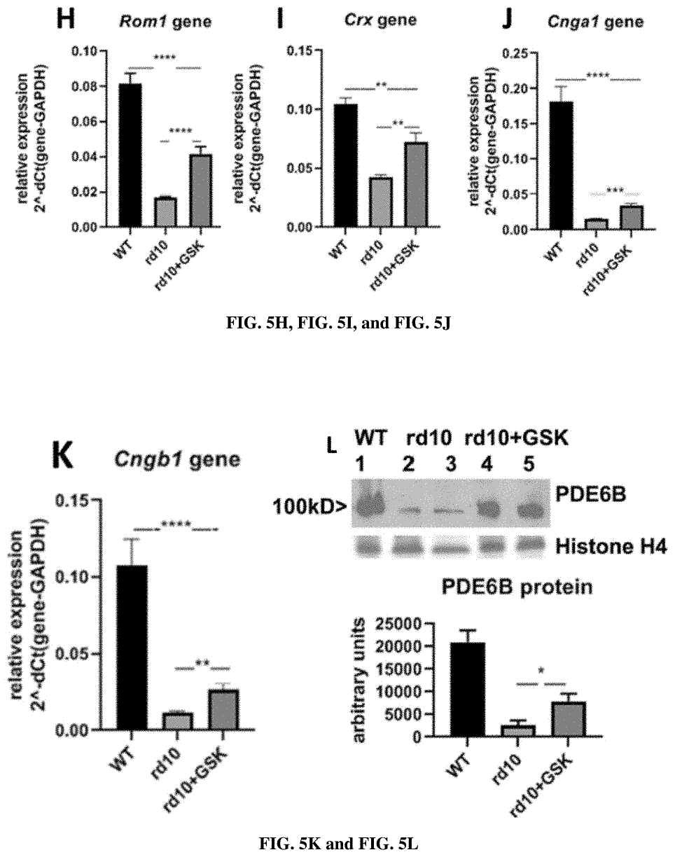

First, changes in rod photoreceptor specific genes were analyzed (FIG. 5A) including a) genes expressed early in development, such as Rom1 and Neurod1, b) genes expressed in mature rods, such as Rho and Sag, and c) transcription factors, such as Crx and Nrl. The treatment of rd10 mice with LSD1 inhibitors TCP, or GSK increased expression of almost all rod genes in retina relative to GAPDH, confirming the RNA-seq results. In comparison, there were no changes in rod photoreceptor genes expression in retina in WT mice treated with TCP (data not shown) or GSK (FIG. 5A).

With GSK treatment, expression of one group of rod specific genes, Rho, Prph2, Nr2e3, Nrl, and Pde6b, was increased dramatically and reached WT level (FIG. 5B-5K). Expression of other rod specific genes, Sag, Rom1, Crx, Cnga1 and Cngb1 was increased, but not to the WT level (FIG. 5B-5K).

The effects of treatment with the HDAC1 inhibitor romidepsin were also analyzed in rd10 mice. This compound had the strongest effect on early rod genes, but very little effect on most late rod genes or TF (FIG. 5A).

While the expression of the Pde6b gene was maintained under LSD1 and HDAC1 inhibition (FIG. 5A and RNA-seq data), this measurement was of RNA and not protein. Western blots were used to demonstrate a three-fold increase of Pde6b protein in retinas treated with inhibitor GSK (FIG. 5L).

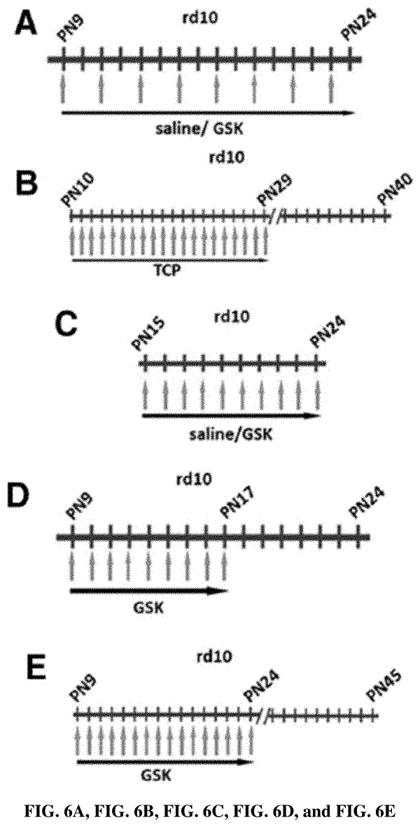

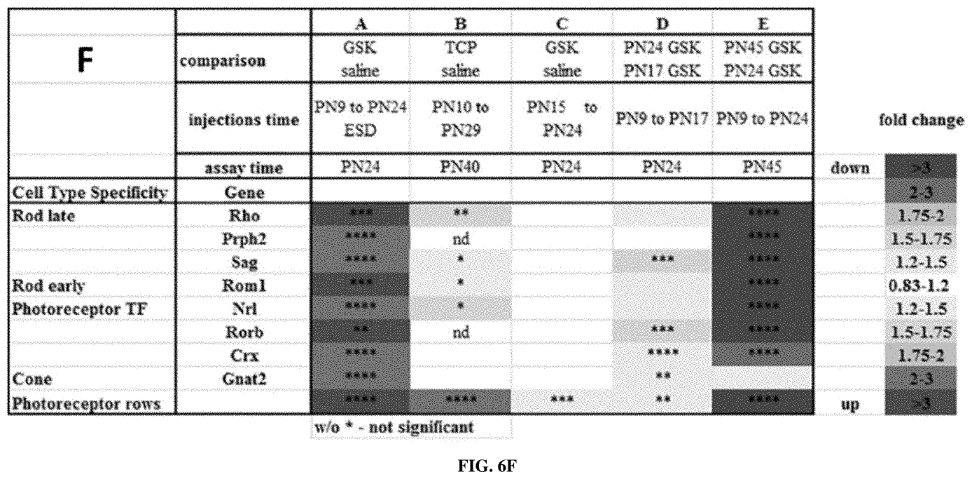

Additionally, rod-specific genes changes when studied at alternative time windows of i.p. injection with epigenetic modulators were examined (FIG. 6, compared with FIG. 2). The changes in expression of markers were closely correlated with preservation of OS and rods in ONL after treatment for rd10 (FIG. 6), for example, for Rho level correlation coefficient is r²=0.92 between number of rod row in retina and Rho expression (data not shown). Thus, the changes in rod photoreceptor genes expression are probably a reflection of the number of rods preserved in the treated rd10 mice.

Changes in Cone Photoreceptor Gene Expression.

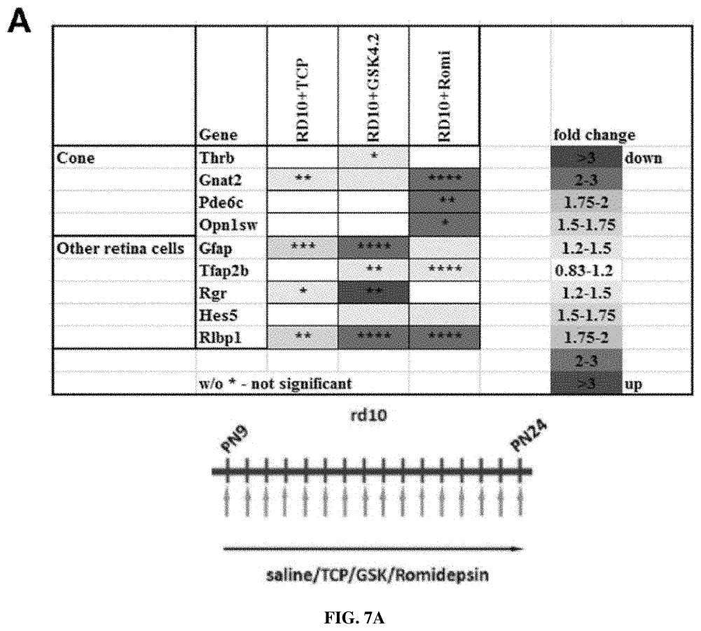

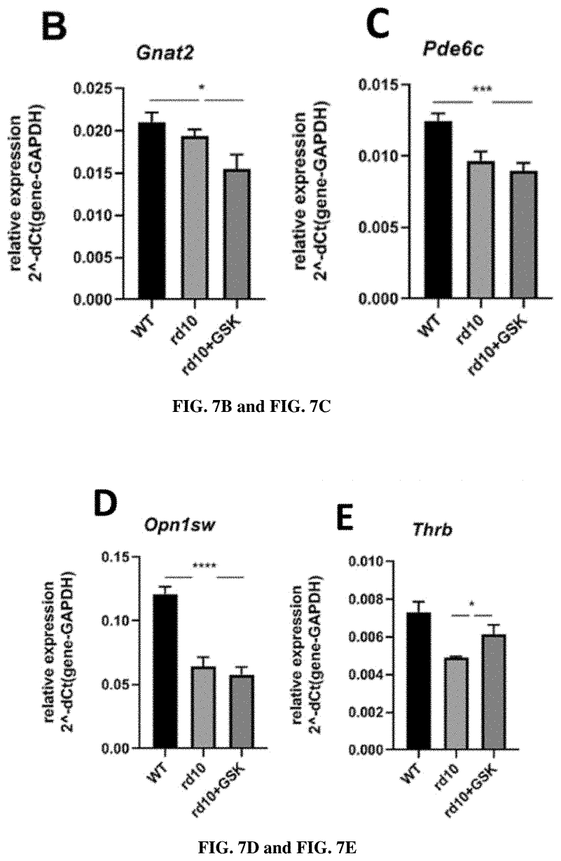

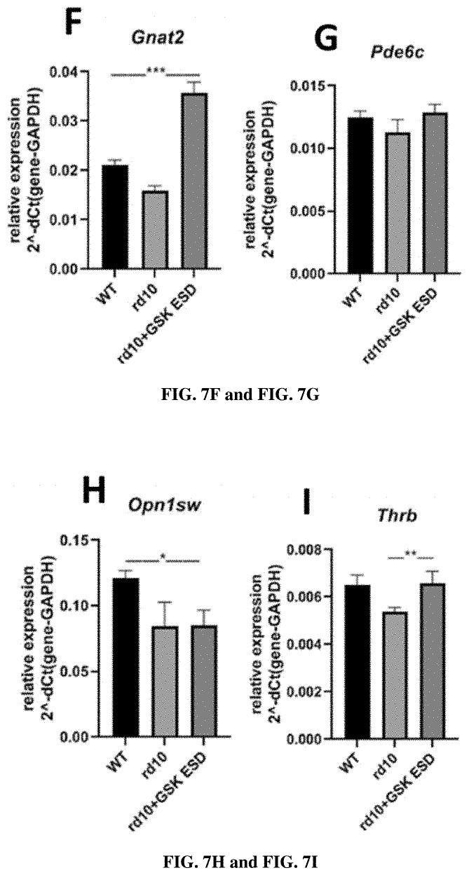

From the RNA-seq data, while expression of genes specific for rod photoreceptors were returned to normal expression levels in rd10+GSK retina, most genes specific for cone photoreceptors, including Opn1sw, Opn1rnw, Thrb, Jam3, Pde6c, Pde6h, Otop3, Gnat2 did not show changes that met the threshold criteria in either rd10 vs WT or in rd10 vs rd10+GSK. This was studied in more detail using RT-PCR. Treatment of rd10 mice with epigenetic inhibitors had small but detectable effects on expression of cone genes (FIG. 7A). In general, expression of cone markers was slightly lower in rd10 at PN24 than in WT retina and treatment with GSK did not change it (FIG. 7B). Some cone genes were slightly up-regulated (Thrb or Gnat2) in rd10 treated with TCP or GSK, but down-regulated following romidepsin treatment (FIG. 7A). The changes in cone-specific genes were estimated under ESD treatment and showed, that except for S-opsin, expression of all other cone genes was increased to WT levels (FIG. 7C), and thus demonstrating that proper GSK treatment not only had neuroprotective effect on rod photoreceptors, but also helped preserve cones.

Changes in Gene Expression of Other Retina Cell Types.

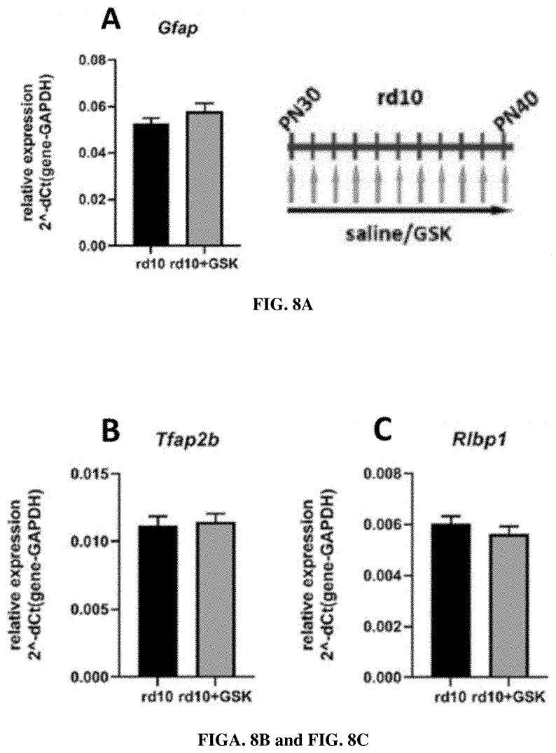

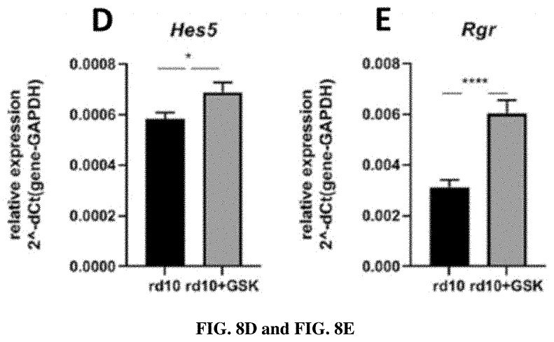

RNA-seq data demonstrated that markers for other retina cell type did not show changes that met the threshold criteria in either rd10 vs WT or in rd10 vs rd10+GSK, except two markers of amacrine cells Clrn1 and Efemp1 that were up-regulated in rd10 and down-regulated in under GSK inhibition. According to qRT-PCR expression of most other retina cell type markers examined appeared to be decreased especially under GSK and romidepsin inhibition, with the exception of Rgr and Hes5 genes (FIG. 7A). GSK treatment of rd10 mice caused an increase in Rgr and Hes5 expression (FIGS. 7A and 8), corroborating the RNA-seq data. This shows that LSD1 inhibition has specific effects on the expression of these genes.

Decreased expression of gene-markers of non-photoreceptor retinal cell types could be either because epigenetic inhibitors are harmful for INL cells or as a result of preserving number of photoreceptors and decreasing percentage of others cell types in rd10 retinas. To distinguish between these two possibilities, rd10 retinas were treated with GSK at later stages from PN30 till PN40, when retinas in rd10 mice consists mostly of INL cell types (FIG. 8). Gene expression did not change, showing that epigenetic inhibitors are not harmful for retina cells in INL, but the increase in proportion of photoreceptors in retina leads to an apparent decrease in relative expression of specific genes from other retina cell types.

LSD1 and HDAC1 Inhibitors Decrease Cell Death, Gliosis, and Inflammation.

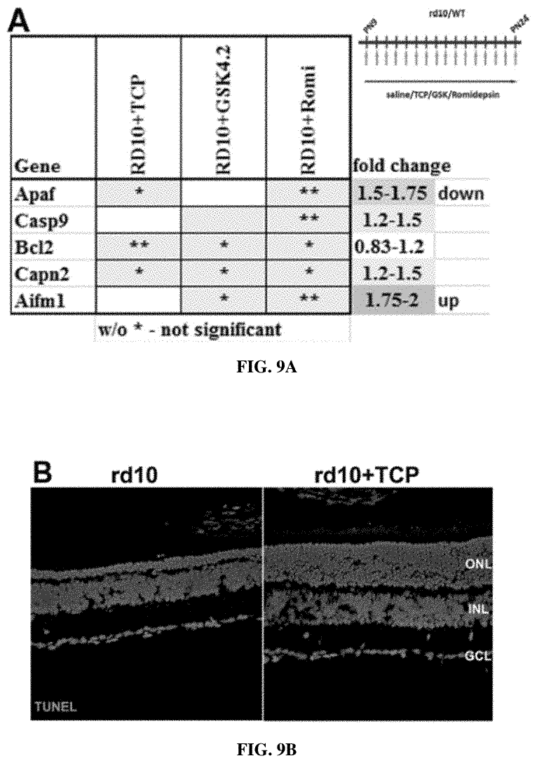

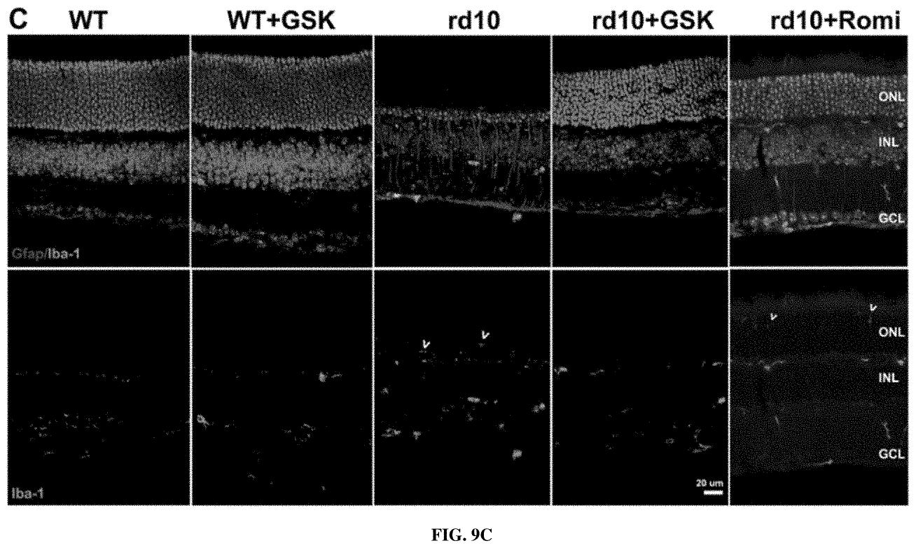

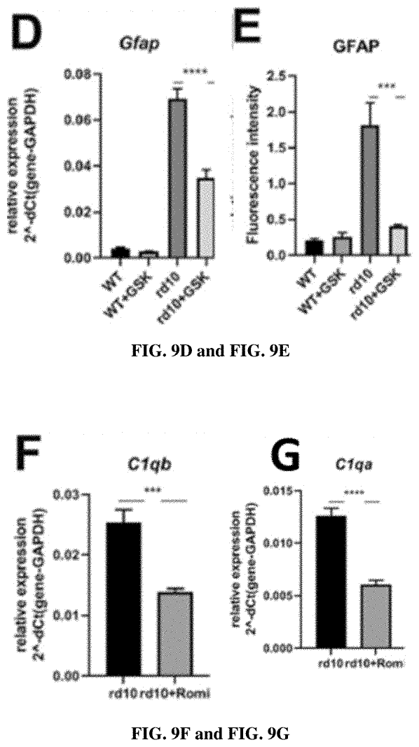

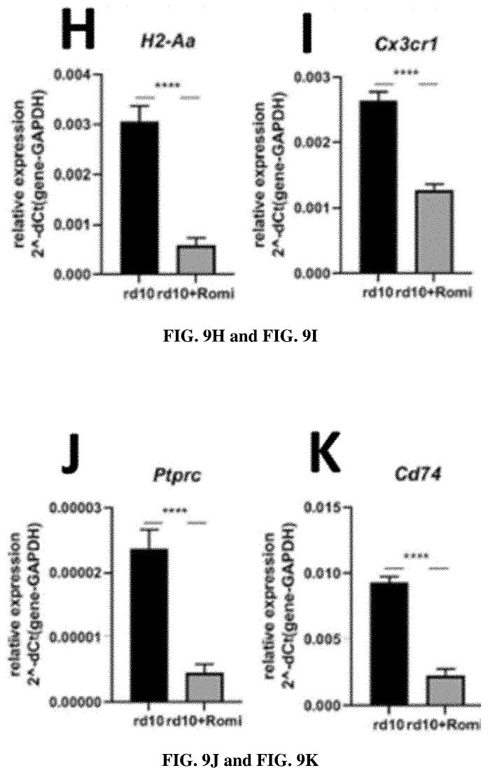

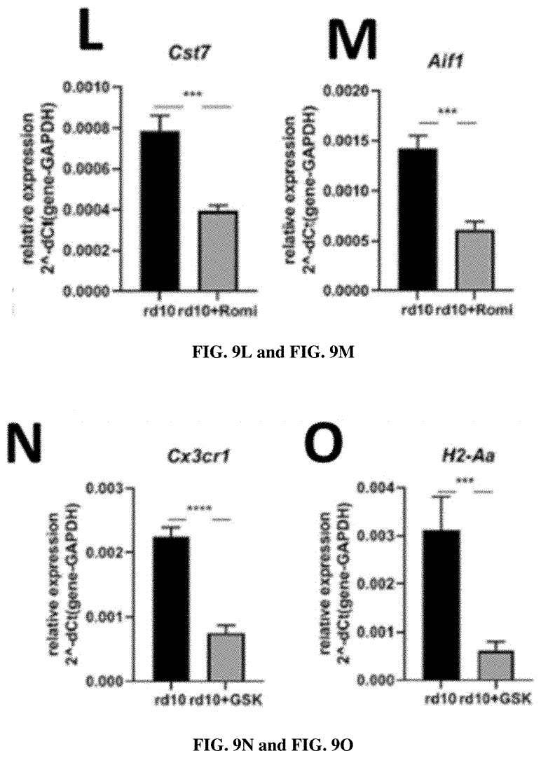

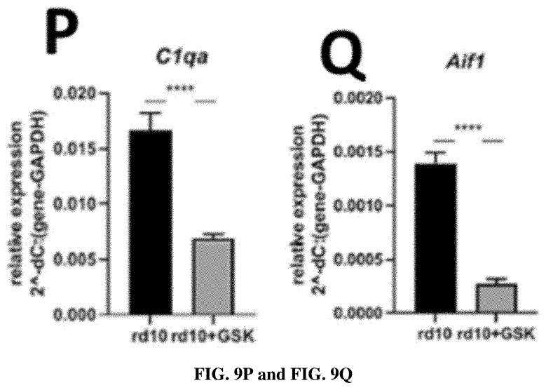

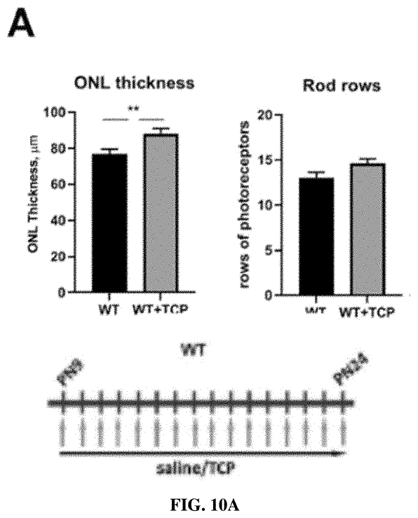

First the amount of cell death in control rd10 was compared to rd10 treated with TCP by TUNEL staining. In both cases only a very small number of dying cells were detected with no significant difference between them (FIG. 9B). Different mechanisms of rod photoreceptors death have been suggested in the literature, so several genes were assayed that allowed us broader understanding of processes occurring in the retina during treatment with epigenetic inhibitors GSK and romidepsin. Expression of apoptotic specific genes Bcl2, Apaf1, and Casp9 was slightly decreased (FIG. 9A). Similarly, expression of the calcium-activated protease calpain 2 (Capn2), and its substrate apoptosis-inducing factor (Aifm1), was somewhat decreased in rd10 mutant mice treated with epigenetic inhibitors (FIG. 9A). The RNA-seq data identified only small number of genes participating in different pathways of cell death that were upregulated in rd10, including Capn9, Casp1, 4, 8, 12, AO (IBA1 protein), Ripk3 and Mlkl; some of these were downregulated under GSK inhibition (Capn9, Casp1, 12, AifI (IBA1 protein)).

GFAP is a marker of both Muller glia and astrocytes, and increased levels of GFAP expression is characteristic of gliosis, a glial response to any damage in the nervous system. According to the RNA-seq data Gfap was increased in rd10 and then decreased under GSK inhibition, but this decrease did not reach our threshold conditions. Both gene expression of Gfap (FIG. 7A and FIG. 9D), and immunofluorescence labeling of GFAP protein (FIGS. 9C and 9E) demonstrated upregulation in rd10 retinas relative to WT, and reductions in Muller cell activation of GSK-treated retinas. similar results were obtained with TCP treatment (data not shown). Retina sections were also labeled for IBA1 (AO gene), a marker for microglia. IBA1 labeling was significantly increased in multiple layers of rd10 as compared with WT and, while the labeling was decreased by GSK/TCP treatment, the levels remained above WT (FIG. 9C and data not shown). This mirrors the increase and decrease in Aif1 RNA that was detected by RNA-seq (see above) and qRT-PCR (FIG. 9G). Thus, rd10 both Muller cells and microglia show responses and that treatment with inhibitors leads to decrease in gliosis in Muller cells.

Since inhibition of HDAC1 with romidepsin also preserved retina from degeneration in rd10 mice (FIG. 1 and FIG. 5A), it was examined whether it exerted the same effects on inflammatory genes as LSD1 inhibition. Following romidepsin treatment the expression level of Gfap did not change (FIG. 7A). Similarly, immunofluorescence labeling for GFAP and IBA1 (AO gene) did not demonstrate significant differences between rd10 and rd10+romidepsin retinas (FIG. 9C and data not shown). Expression levels for inflammatory markers were down regulated under HDAC1 inhibition, including pan-leucocytes marker Ptprc (Cd45), pan-microglia marker C1qa, activated microglia marker C1gb, a marker of activated resident microglia Cst7, markers of infiltrating macrophages H2-Aa and Cd74, chemokine receptor Cx3cr1 and Aif1 (IBA1 protein)(FIG. 9F). RNA levels of H2-Aa, C1qa, Aif1 and Cxc3cr1 were also all reduced by treatment with GSK (FIG. 9G). Thus, romidepsin treatment inhibited microglia activation on the level of RNA in the same way as GSK but was less powerful at the protein level for proteins such as GFAP and IBA1.

LSD1 Inhibition Promotes Epigenetic Changes that Create More Open and Accessible Chromatin in Rod Nuclei and Making Rods Less Mature.

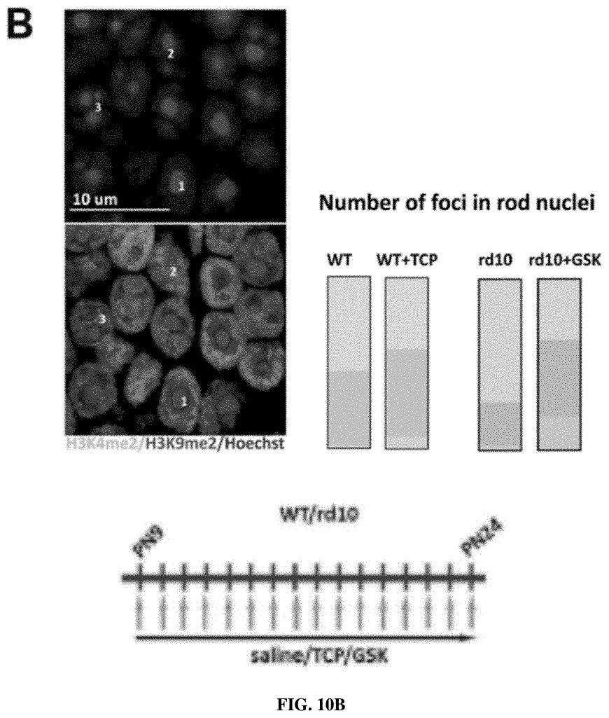

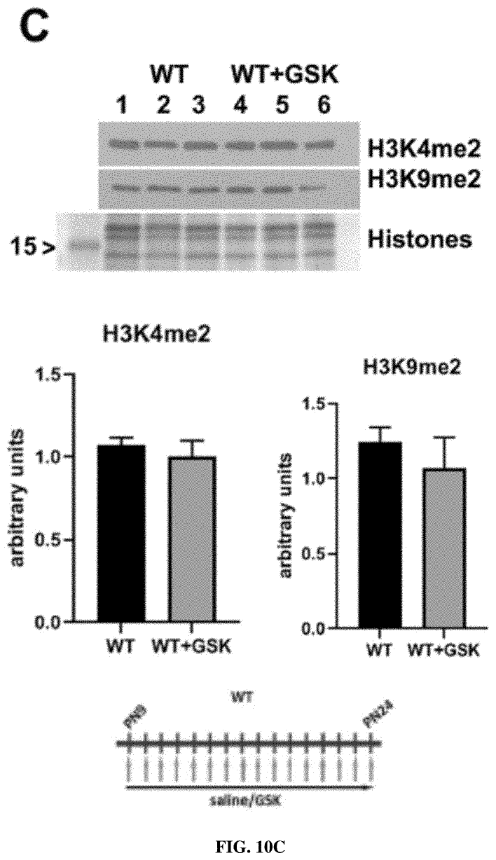

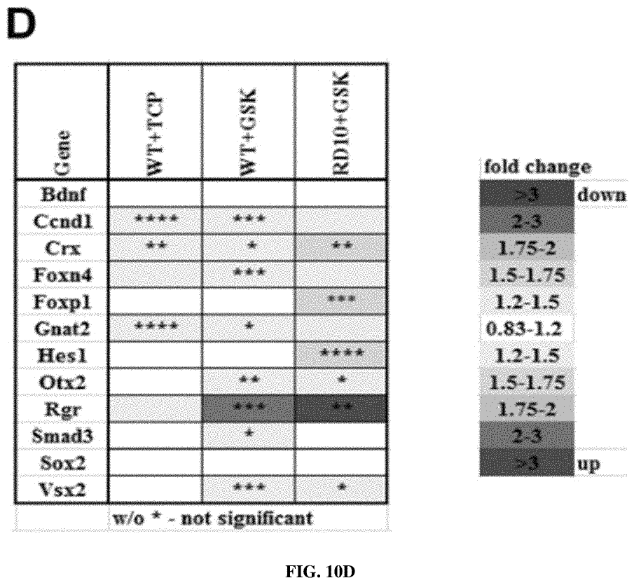

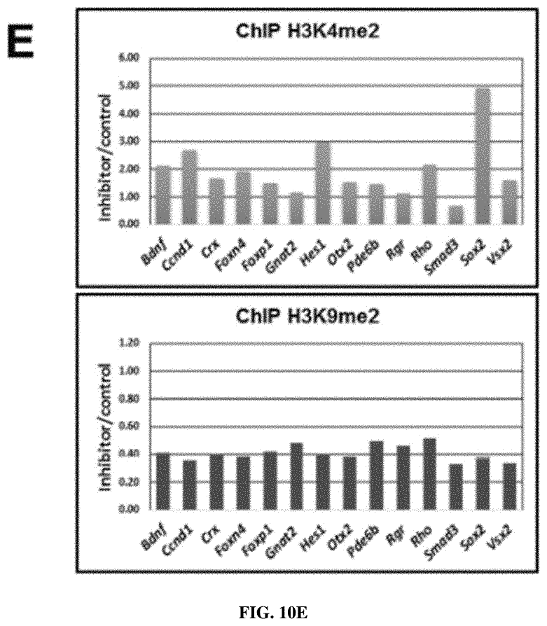

The range of gene expression changes and the changes in epigenetic marks shows that epigenetic inhibitor treatments was having effects on chromatin structure. It is noted that the number of rows of rods in WT did not change but the ONL thickness was significantly increased (FIG. 10A) after treating WT mice with TCP, as a control, showing fewer compact nuclei and cell bodies. During retina postnatal maturation mouse rod photoreceptor nuclei undergo dramatic transformation when foci or chromocenters of heterochromatin that at PN1 were seen located mostly at the nuclear periphery, around PN15 began to relocate toward the center of the rod nuclei and started to fuse together, so that at PN28 rod nuclei have mostly two foci and in adult eyes (PN56) one big focus of heterochromatin occupied most of the nuclear volume pushing euchromatin out to the nuclear periphery. The number of heterochromatin foci in rod nuclei was calculated to estimate the level of rod photoreceptor maturation in retinas of animals treated with LSD1 inhibitors (FIG. 10B). In WT animals retina at PN24 55% rod nuclei have 1 focus, 43% have 2 foci and only 2% have 3 foci, while in WT animals treated with TCP fewer rod nuclei have one focus (42%), 51% have two foci and 7% have nuclei with 3 foci. In untreated rd10 animals proportion of nuclei with one focus in the remaining rod photoreceptors is even bigger than in WT, 72%, and only 26% had two foci and 3% had three foci nuclei. Rod photoreceptor nuclei in rd10 retinas treated with GSK have 35% with one focus, 45% with two and 20% with three foci (FIG. 10B). This demonstrates that treatment with LSD1 inhibitors leads to slower rod photoreceptor maturation and less compact heterochromatin in their nuclei.

The enzymatic activities of LSD1 and HDAC1 remove active epigenetic methylation and acetylation marks and inhibition of these enzymes should leave these active epigenetic marks intact. To test whether the inhibitors could change epigenetic profiles of retina WT mice treated with TCP or GSK were used, but not rd10 mice, because rd10 retinas undergoing degeneration will have changes in cell type composition that will influence epigenetic profile dramatically. Global levels of H3K4me2 did not change significantly following treatment with inhibitors as judged by Western blots (FIG. 10C). Additionally, facultative heterochromatin marks of H3K9me2 were checked that has been considered a substrate for LSD1. No changes in global levels of H3K9me2 were seen in retinas of treated mice (FIG. 10C).

Several transcription factors (TF) that participate in retina development and maturation showed slightly increased expression in WT retinas treated with TCP or GSK (FIG. 10D). Chromatin Immunoprecipitation (ChIP) was used to measure if there were changes in H3K4me2 marks on gene promoters or regulatory elements under GSK inhibition in WT mice for these genes. GSK-mediated LSD1 inhibition caused increase in the methylation of H3K4 at the promoters/regulatory elements of most of these genes, for example of cell cycle and progenitor genes Ccnd1, Sox2 and Hes1, as well as of rod specific genes Rho and Pde6b (FIG. 10E, upper panel). Although the overall level of H3K9me2 as detected on Western blots (FIG. 10C) did not change, probably because much of the H3K9me2 signal comes from mouse major satellite repeats, chromatin immunoprecipitation demonstrated that the regulatory elements of progenitor TF and rod specific genes were losing this inhibitory mark (FIG. 10E, bottom panel) in retina under GSK inhibition. This shows that neuroprotective treatment with these epigenetic inhibitors leads to less mature and/or less compact, but more open and accessible chromatin.

### Discussion

Retinitis Pigmentosa is a very heterogeneous disease with numerous different mutations and pathways leading to retina degeneration, showing the most fruitful way to fight this disease needs to be gene-independent. A number of molecular pathways have been implicated in triggering cell death of rod photoreceptors in RP. These include dysregulation of cGMP- and Ca²⁺ signaling, insufficient proteasomal activity and accumulation of mis-folded proteins, oxidative stress, and inflammation.

It was demonstrated that inhibitors of two histone modification erasers, LSD1 and HDAC1, in a mouse model of RP, rd10, led to rod photoreceptor preservation (FIGS. 1B, 1D, and 1E), retained expression of rod-specific genes (FIGS. 4 and 5) and maintenance of visual function (FIG. 2F). The effect of inhibiting LSD1 was also tested on degeneration in mouse with more rapid retina degeneration, rd1 mutant, but it was difficult to find an effective time frame for injection of epigenetic inhibitors due to the rapid deterioration of the retina before photoreceptors have fully developed. Two molecular mechanisms that account for this neuroprotective effect of the epigenetic inhibitors were identified. First, acting on histone targets in photoreceptors, they increased accessibility of chromatin and upregulated neuroprotective genes (FIG. 4B and FIG. 10). Second, acting on non-histone targets of LSD1 and HDAC1 in microglia, resident and infiltrating immune cells, they inhibit transcription of inflammatory genes and inflammation (FIG. 4C-4H and FIG. 9C-9G, Tables 3, 4). Both mechanisms result in enhanced survival of functional rod photoreceptors.

Inhibition of the enzymatic activity of LSD1 and HDAC1 toward histone targets caused retention of active epigenetic marks in the genome of rod photoreceptors (FIG. 10E), epigenetic changes in heterochromatin organization of rod nuclei (FIG. 10B) and an increase in the proportion of open and accessible chromatin. The morphological changes in chromatin condensation observed were supported by measurements of increased ONL thickness (FIG. 10A) and upregulated expression of progenitor/cell cycle genes (FIG. 10D). A cluster of upregulated genes belong to the WNT pathway and have been shown to play a role in neuroprotection. The pleiotropic changes in gene expression induced by the epigenetic inhibitors support a change in cell state or metabolism that allows survival and function of rod photoreceptors without deleterious changes in other retinal cell types.

Susceptibility to cell death (and degeneration) or to re-entry into the cell cycle (and malignant transformation) are inversely correlated and the underlying mechanism determining these two opposite cellular properties is epigenome organization. Cells with more open active chromatin organization can more easily survive change in cellular homeostasis in response to stress, but such cells are prone to cancerous transformation. Cells with more closed heterochromatic nuclear organization are less susceptible to malignancy but have a lower ability to survive stress and make them predisposed to degeneration and cell death. Mature rod photoreceptors, like most neurons, belong to the second group of cells, and have a uniquely closed chromatin organization. This example shows that loosening or decondensing heterochromatin in rods can reduce degeneration and allow better survival of rod photoreceptors under stress conditions but, based on Ki67 labeling, do not demonstrate re-entry into cell cycle (data not shown).

The second molecular mechanism detected in rd10 was a dramatic decrease in inflammatory markers. Retina genes expression profiles were compared by RNA-seq in rd10 versus WT (FIG. 4C) and demonstrated a dramatic up-regulation of several inflammatory pathways (FIGS. 4E and 4G, Table 3) that was reversed by treatment with LSD1 inhibitor GSK (FIGS. 4D, 4F, and 4H). LSD1 participates in a signaling cascade (PKCα-LSD1-NF-kB) and demethylates one of the subunits of the NF-kB complex, p65 (gene Rela), enhancing its ability to activate expression of NF-kB target genes in the inflammatory response during sepsis and colitis. It was contemplated that a similar pathway is activated during retina degeneration and that LSD1 inhibition blocks this pathway and abrogates inflammation. The findings herein support a role for immune responses in both mouse models of RP and in human patients.

Interestingly, HDAC1 inhibition were found to have a similar inhibitory effect on transcription of inflammatory genes as LSD1 inhibition. While LSD1 and HDAC1 are known to interact synergistically in the nucleus to change patterns of epigenetic histone modifications, there is less evidence for such an interaction for non-histone targets. Acetylation of p65 reduces its binding to DNA in promoter regions of inflammatory genes. Thus, HDAC inhibition with romidepsin could lead to higher acetylation of p65 and inhibition of transcription regulated by the NF-kB pathway.

A number of previous studies have tested epigenetic modifiers as possible therapeutics for retinitis pigmentosa. Some of these have focused on the neuroprotective effects of blocking HDAC activity by such non-selective inhibitors as TSA, VPA and sodium butyrate, all of which have some protective effect on RP. The broad-spectrum HDAC inhibitor VPA has been tested as a therapeutic agent for retinal degeneration with mixed results. Recently, selective inhibitors that are specific for subclasses of HDACs were tested to prevent neurodegeneration, for example, specific inhibition of HDAC3 by RGFP966 protected against RGC death in models of optic nerve injury.

Additional treatments with other modifiers that lessen chromatin compaction is just beginning. Inhibition of DNA methylation in rd1 mouse with decitabine resulted in reduction of photoreceptor loss. Inhibition of PRC2 deposition of the repressive chromatin mark H3K27me3 by DZNep led to delays in retinal degeneration in rd1 mice. BMI1 is a component of another polycomb repressive complex 1 (PRC1) and also performs chromatin compaction by activating PRC2 complex. Knocking out BMI1 resulted in photoreceptor survival in rd1 retina. Pharmacological inhibition of HDAC11 and SUV39H2, that made chromatin more open and accessible, ameliorated age-related macular degeneration. Whether some or all of these compounds also inhibit inflammation is not known.

The Class I HDAC inhibitor romidepsin was tested and has been approved for use in treating peripheral and cutaneous T-cell lymphoma. Romidepsin was moderately effective at preventing rod degeneration in rd10 mice and increased the levels of expression of mostly early rod's genes, but not photoreceptor TFs or later rod and cone genes, probably because it caused higher decondensation of chromatin than LSD1 inhibitors. Animals treated with romidepsin, however, showed poorer weight gain and were less active than those treated with other agents. While intraocular or topical treatments with romidepsin or related compounds might overcome some of the systemic negative effects, this data shows that there are better systemic treatments.

These results clearly show that inhibiting LSD1 with either TCP or GSK led to increased survival of photoreceptors and no deleterious effects on other retinal cells, with decreased expression of markers of inflammation, cell death and gliosis. Interestingly, the treatments were able to stop degeneration at whatever point they were started, but the degeneration resumed when the treatments ceased. The best result for retina preservation in rd10 mice was obtained with the specific LSD1 inhibitor, GSK. Though a better response was detected at the higher dose used, drug application every two days was just as effective and had fewer side effects in that animals showed more normal gain in body weight and were more active.

The heterogeneity of RP has hindered the development of general therapies. It has been demonstrated that pharmacological manipulation of LSD1 and HDAC1 alters the epigenetic landscape in ways that lessen the impact of deleterious mutations and inflammation and allows extended survival of rod photoreceptors.

Herein, it is shown that epigenetic modifiers can effectively treat RP because of their dual action. By reducing inflammation, they provide an environment in which a more open chromatin structure can allow utilization of a wider array of homeostatic mechanisms to survive and prevent cell death pathway activation.

### Example 2: Treating Neurological Disorders by Epigenetic Modifiers

Treating neurological disorders by epigenetic modifiers that act by changing chromatin structure and gene transcription without altering the genome DNA is a powerful approach that can be applied to genetic disorders manifested in mature tissues for which genome editing is not an option.

These data have strongly supported the concept that epigenetic modifiers causing chromatin decondensation can prevent degeneration in a wide range of retinal diseases. Chromatin condensation occurs through two primary mechanisms: 1) removing active epigenetic marks, such as H3K4 methylation or acetylation of histones on multiple lysine residue, and/or 2) deposition of repressive epigenetic marks, such as methylation of H3K27, H3K9 and DNA methylation.

To increase chromatin accessibility, inhibitors of both classes of mechanism can be used. It has been demonstrated that lessening chromatin condensation leads to preservation of rod photoreceptor and improves vision in mouse model of Retinitis pigmentosa, rd10 mice. Several inhibitors of these mechanisms have been used. LSD1 demethylates H3K4me2, 3, and treating rd10 mice LSD1 inhibitors trans-2-phenylcyclopropylamine (TCP) and GSK2879552 improve rod photoreceptor survival. Romidepsin, an inhibitor of histone deacetylases (HDACs), has the same effect.

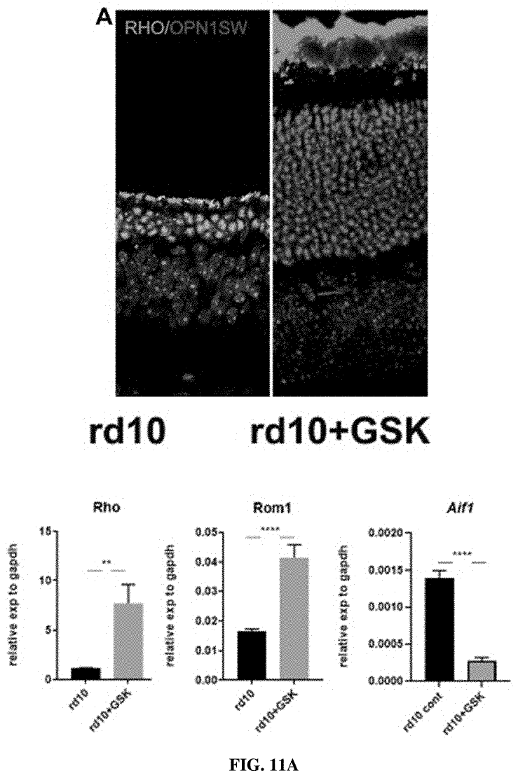

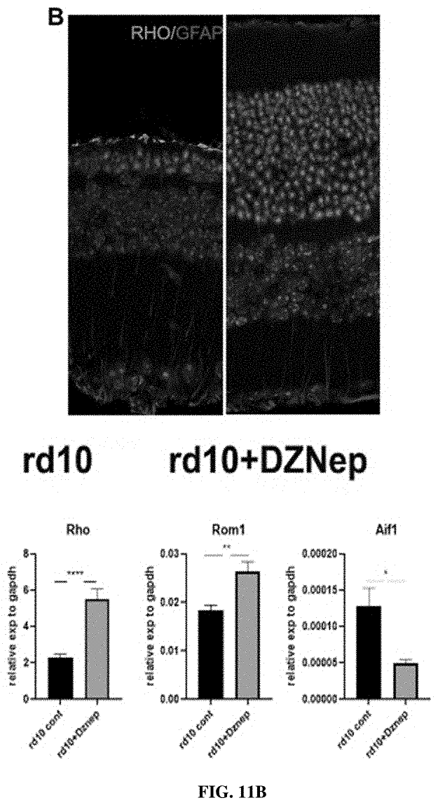

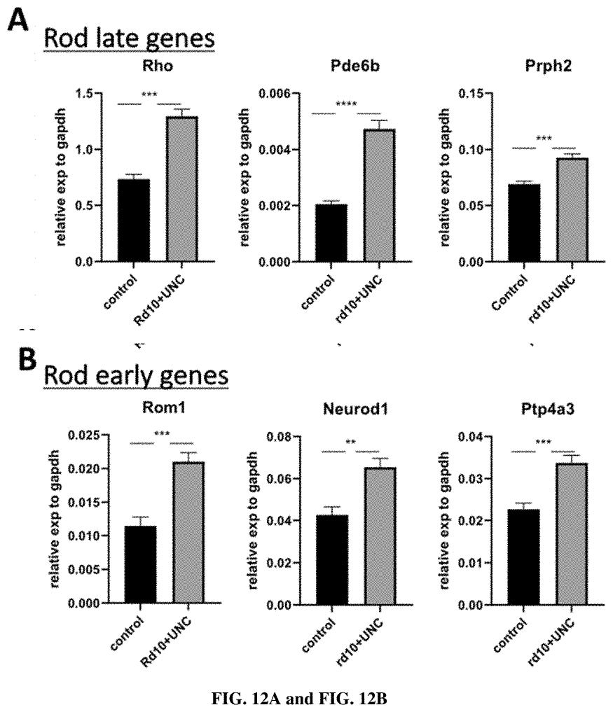

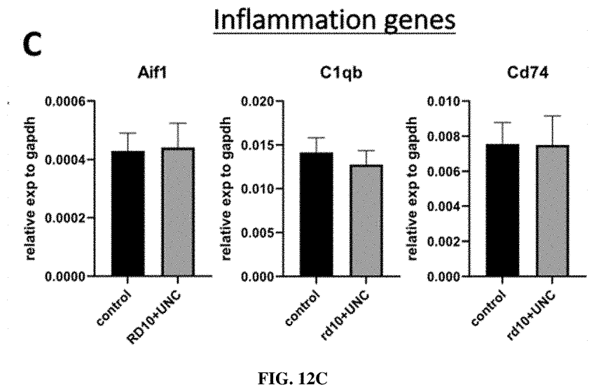

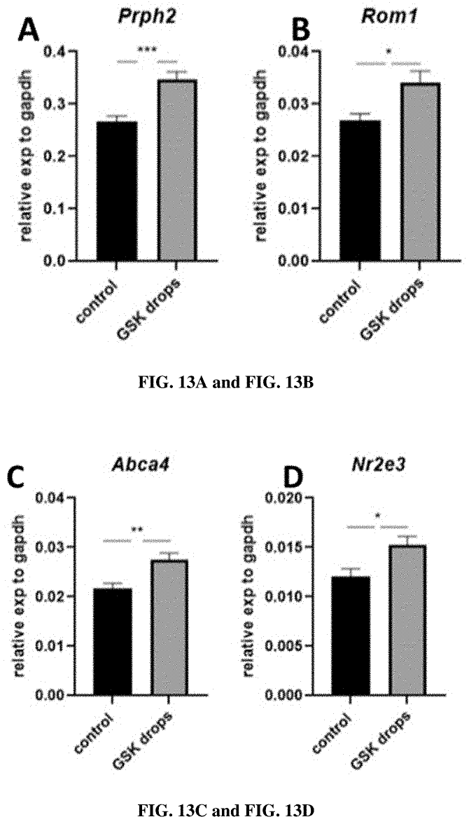

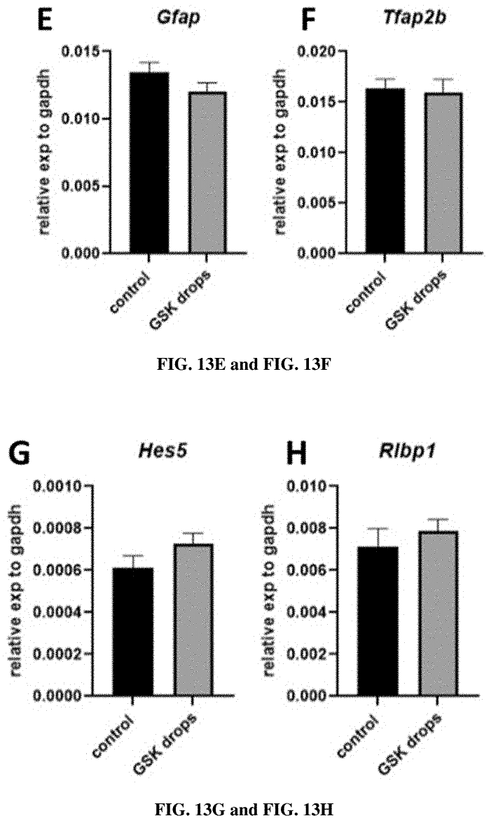

FIGS. 11A-13H further demonstrates the effects of epigenetic modifiers.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### REFERENCES

Adamo A, Sesé B, Boue S, Castaño J, Paramonov I, Barrero M J, Izpisua Belmonte J C (2011) LSD1 regulates the balance between self-renewal and differentiation in human embryonic stem cells. Nat Cell Biol 13:652-659.
Aldiri I et al. (2017) The Dynamic Epigenetic Landscape of the Retina During Development, Reprogramming, and Tumorigenesis. Neuron 94:550-568.e510.
Arango-Gonzalez B, Trifunovic\acute{ } D, Sahaboglu A, Kranz K, Michalakis S, Farinelli P, Koch S, Koch F, Cottet S, Janssen-Bienhold U, Dedek K, Biel M, Zrenner E, Euler T, Ekström P, Ueffing M, Paquet-Durand F (2014) Identification of a common non-apoptotic cell death mechanism in hereditary retinal degeneration. PLoS One 9:e112142.
Barnstable C J (1980) Monoclonal antibodies which recognize different cell types in the rat retina. Nature 286:231-235.
Berner A K, Kleinman M E (2016) Therapeutic Approaches to Histone Reprogramming in Retinal Degeneration. Adv Exp Med Biol 854:39-44.
Bishton M J, Harrison S J, Martin B P, McLaughlin N, James C, Josefsson E C, Henley K J, Kile B T, Prince H M, Johnstone R W (2011) Deciphering the molecular and biologic processes that mediate histone deacetylase inhibitor-induced thrombocytopenia. Blood 117:3658-3668.
Candido E P, Reeves R, Davie J R (1978) Sodium butyrate inhibits histone deacetylation in cultured cells. Cell 14:105-113.
Chang B, Hawes N L, Hurd R E, Davisson M T, Nusinowitz S, Heckenlively J R (2002) Retinal degeneration mutants in the mouse. Vision Res 42:517-525.
Chang B, Hawes N L, Pardue M T, German A M, Hurd R E, Davisson M T, Nusinowitz S, Rengarajan K, Boyd A P, Sidney S S, Phillips M J, Stewart R E, Chaudhury R, Nickerson J M, Heckenlively J R, Boatright J H (2007) Two mouse retinal degenerations caused by missense mutations in the beta-subunit of rod cGMP phosphodiesterase gene. Vision Res 47:624-633.
Charish J, Shabanzadeh A P, Chen D, Mehlen P, Sethuramanujam S, Harada H, Bonilha V L, Awatramani G, Bremner R, Monnier P P (2020) Neogenin neutralization prevents photoreceptor loss in inherited retinal degeneration. J Clin Invest 130:2054-2068.
Chuang D M, Leng Y, Marinova Z, Kim H J, Chiu C T (2009) Multiple roles of HDAC inhibition in neurodegenerative conditions. Trends Neurosci 32:591-601.
Cisneros E, di Marco F, Rueda-Carrasco J, Lillo C, Pereyra G, Martín-Bermejo M J, Vargas A, Sanchez R, Sandonís Á, Esteve P, Bovolenta P (2020) Sfrp1 deficiency makes retinal photoreceptors prone to degeneration. Sci Rep 10:5115.
Clemson C M, Tzekov R, Krebs M, Checchi J M, Bigelow C, Kaushal S (2011) Therapeutic potential of valproic acid for retinitis pigmentosa. Br J Ophthalmol 95:89-93.
Dyer M A (2016) Lessons from Retinoblastoma: Implications for Cancer, Development, Evolution, and Regenerative Medicine. Trends Mol Med 22:863-876.
Farinelli P, Perera A, Arango-Gonzalez B, Trifunovic D, Wagner M, Carell T, Biel M, Zrenner E, Michalakis S, Paquet-Durand F, Ekström P A (2014) DNA methylation and differential gene regulation in photoreceptor cell death. Cell Death Dis 5:e1558.
Ferreira R C, Popova E Y, James J, Briones M R, Zhang S S, Barnstable C J (2017) Histone Deacetylase 1 Is Essential for Rod Photoreceptor Differentiation by Regulating Acetylation at Histone H3 Lysine 9 and Histone H4 Lysine 12 in the Mouse Retina. J Biol Chem 292:2422-2440.
Gargini C, Terzibasi E, Mazzoni F, Strettoi E (2007) Retinal organization in the retinal degeneration 10 (rd10) mutant mouse: a morphological and ERG study. J Comp Neurol 500:222-238.
Gupta N, Brown K E, Milam A H (2003) Activated microglia in human retinitis pigmentosa, late-onset retinal degeneration, and age-related macular degeneration. Exp Eye Res 76:463-471.
Hart A W, McKie L, Morgan J E, Gautier P, West K, Jackson I J, Cross S H (2005) Genotype-phenotype correlation of mouse pde6b mutations. Invest Ophthalmol Vis Sci 46:3443-3450.
Holly V L, Widen S A, Famulski J K, Waskiewicz A J (2014) Sfrp1a and Sfrp5 function as positive regulators of Wnt and BMP signaling during early retinal development. Dev Biol 388:192-204.
Hughes A E, Enright J M, Myers C A, Shen S Q, Corbo J C (2017) Cell Type-Specific Epigenomic Analysis Reveals a Uniquely Closed Chromatin Architecture in Mouse Rod Photoreceptors. Sci Rep 7:43184.
Iraha S, Hirami Y, Ota S, Sunagawa G A, Mandai M, Tanihara H, Takahashi M, Kurimoto Y (2016) Efficacy of valproic acid for retinitis pigmentosa patients: a pilot study. Clin Ophthalmol 10:1375-1384.
Iribarne M, Masai I (2018) Do cGMP Levels Drive the Speed of Photoreceptor Degeneration? Adv Exp Med Biol 1074:327-333.
Jain S, Jirau-Serrano X, Zullo K M, Scotto L, Palermo C F, Sastra S A, Olive K P, Cremers S, Thomas T, Wei Y, Zhang Y, Bhagat G, Amengual J E, Deng C, Karan C, Realubit R, Bates S E, O'Connor O A (2015) Preclinical pharmacologic evaluation of pralatrexate and romidepsin confirms potent synergy of the combination in a murine model of human T-cell lymphoma. Clin Cancer Res 21:2096-2106.
Kang S, Larbi D, Andrade M, Reardon S, Reh T A, Wohl S G (2020) A Comparative Analysis of Reactive Müller Glia Gene Expression After Light Damage and microRNA-Depleted Müller Glia-Focus on microRNAs. Front Cell Dev Biol 8:620459.
Kerenyi M A, Shao Z, Hsu Y J, Guo G, Luc S, O'Brien K, Fujiwara Y, Peng C, Nguyen M, Orkin S H (2013) Histone demethylase Lsd1 represses hematopoietic stem and progenitor cell signatures during blood cell maturation. Elife 2:e00633.
Kiernan R, Brés V, Ng R W, Coudart M P, El Messaoudi S, Sardet C, Jin D Y, Emiliani S, Benkirane M (2003) Post-activation turn-off of NF-kappa B-dependent transcription is regulated by acetylation of p65. J Biol Chem 278:2758-2766.
Kim D, Nam H J, Lee W, Yim H Y, Ahn J Y, Park S W, Shin H R, Yu R, Won K J, Bae J S, Kim K I, Baek S H (2018) PKCα-LSD1-NF-κB-Signaling Cascade Is Crucial for Epigenetic Control of the Inflammatory Response. Mol Cell 69:398-411.e396.
   Kumar A, Midha N, Gogia V, Gupta S, Sehra S, Chohan A (2014) Efficacy of oral valproic acid in patients with retinitis pigmentosa. J Ocul Pharmacol Ther 30:580-586.
   Laurent B, Ruitu L, Murn J, Hempel K, Ferrao R, Xiang Y, Liu S, Garcia B A, Wu H, Wu F, Steen H, Shi Y (2015) A specific LSD1/KDM1A isoform regulates neuronal differentiation through H3K9 demethylation. Mol Cell 57:957-970.
   Lobanova E S, Finkelstein S, Li J, Travis A M, Hao Y, Klingeborn M, Skiba N P, Deshaies R J, Arshaysky V Y (2018) Increased proteasomal activity supports photoreceptor survival in inherited retinal degeneration. Nat Commun 9:1738.
   Luu J, Kallestad L, Hoang T, Lewandowski D, Dong Z, Blackshaw S, Palczewski K (2020) Epigenetic hallmarks of age-related macular degeneration are recapitulated in a photosensitive mouse model. Hum Mol Genet.
   McLaughlin M E, Sandberg M A, Berson E L, Dryja T P (1993) Recessive mutations in the gene encoding the beta-subunit of rod phosphodiesterase in patients with retinitis pigmentosa. Nat Genet 4:130-134.
   Mitton K P, Guzman A E, Deshpande M, Byrd D, DeLooff C, Mkoyan K, Zlojutro P, Wallace A, Metcalf B, Laux K, Sotzen J, Tran T (2014) Different effects of valproic acid on photoreceptor loss in Rd1 and Rd10 retinal degeneration mice. Mol Vis 20:1527-1544.
   Mohammad H P et al. (2015) A DNA Hypomethylation Signature Predicts Antitumor Activity of LSD1 Inhibitors in SCLC. Cancer Cell 28:57-69.
   Murakami Y, Ishikawa K, Nakao S, Sonoda K H (2020) Innate immune response in retinal homeostasis and inflammatory disorders. Prog Retin Eye Res 74:100778.
   Newton F, Megaw R (2020) Mechanisms of Photoreceptor Death in Retinitis Pigmentosa. Genes (Basel) 11.
   Norrie J L, Lupo M S, Xu B, Al Diri I, Valentine M, Putnam D, Griffiths L, Zhang J, Johnson D, Easton J, Shao Y, Honnell V, Frase S, Miller S, Stewart V, Zhou X, Chen X, Dyer M A (2019) Nucleome Dynamics during Retinal Development. Neuron 104:512-528.e511.
O'Koren E G, Yu C, Klingeborn M, Wong A Y W, Prigge C L, Mathew R, Kalnitsky J, Msallam R A, Silvin A, Kay J N, Bowes Rickman C, Arshaysky V Y, Ginhoux F, Merad M, Saban D R (2019) Microglial Function Is Distinct in Different Anatomical Locations during Retinal Homeostasis and Degeneration. Immunity 50:723-737.e727.
Oh C, Jeong J, Oh S K, Baek S H, Kim K I (2020) Inhibition of LSD1 phosphorylation alleviates colitis symptoms induced by dextran sulfate sodium. In: BMB Rep, pp 385-390.
Popova E Y, Pinzon-Guzman C, Salzberg A C, Zhang S S, Barnstable C J (2016) LSD1-Mediated Demethylation of H3K4me2 Is Required for the Transition from Late Progenitor to Differentiated Mouse Rod Photoreceptor. Mol Neurobiol 53:4563-4581.
Popova E Y, Grigoryev S A, Fan Y, Skoultchi A I, Zhang S S, Barnstable C J (2013) Developmentally regulated linker histone H1c promotes heterochromatin condensation and mediates structural integrity of rod photoreceptors in mouse retina. J Biol Chem 288:17895-17907.
Popova E Y, Xu X, DeWan A T, Salzberg A C, Berg A, Hoh J, Zhang S S, Barnstable C J (2012) Stage and gene specific signatures defined by histones H3K4me2 and H3K27me3 accompany mammalian retina maturation in vivo. PLoS One 7:e46867.
Power M, Das S, Schütze K, Marigo V, Ekström P, Paquet-Durand F (2020a) Cellular mechanisms of hereditary photoreceptor degeneration—Focus on cGMP. Prog Retin Eye Res 74:100772.
Power M J, Rogerson L E, Schubert T, Berens P, Euler T, Paquet-Durand F (2020b) Systematic spatiotemporal mapping reveals divergent cell death pathways in three mouse models of hereditary retinal degeneration. J Comp Neurol 528:1113-1139.
Prusky G T, Alam N M, Beekman S, Douglas R M (2004) Rapid quantification of adult and developing mouse spatial vision using a virtual optomotor system. Invest Ophthalmol Vis Sci 45:4611-4616.
Riggs M G, Whittaker R G, Neumann J R, Ingram V M (1977) n-Butyrate causes histone modification in HeLa and Friend erythroleukaemia cells. Nature 268:462-464.
Ronning K E, Karlen S J, Miller E B, Burns M E (2019) Molecular profiling of resident and infiltrating mononuclear phagocytes during rapid adult retinal degeneration using single-cell RNA sequencing. Sci Rep 9:4858.
Sancho-Pelluz J, Alavi M V, Sahaboglu A, Kustermann S, Farinelli P, Azadi S, van Veen T, Romero F J, Paquet-Durand F, Ekstrom P (2010) Excessive HDAC activation is critical for neurodegeneration in the rd1 mouse. Cell Death Dis 1:e24.
Schmitt H M, Schlamp C L, Nickells R W (2017) Targeting HDAC3 Activity with RGFP966 Protects Against Retinal Ganglion Cell Nuclear Atrophy and Apoptosis After Optic Nerve Injury. J Ocul Pharmacol Ther.
Shi L, Cui S, Engel J D, Tanabe O (2013) Lysine-specific demethylase 1 is a therapeutic target for fetal hemoglobin induction. Nat Med 19:291-294.
Shi Y J, Matson C, Lan F, Iwase S, Baba T, Shi Y (2005) Regulation of LSD1 histone demethylase activity by its associated factors. Mol Cell 19:857-864.
Solovei I, Kreysing M, Lanctot C, Kosem S, Peichl L, Cremer T, Guck J, Joffe B (2009) Nuclear architecture of rod photoreceptor cells adapts to vision in mammalian evolution. Cell 137:356-368.
Sun G, Alzayady K, Stewart R, Ye P, Yang S, Li W, Shi Y (2010) Histone demethylase LSD1 regulates neural stem cell proliferation. Mol Cell Biol 30:1997-2005.
Tan L, Xing D, Daley N, Xie X S (2019) Three-dimensional genome structures of single sensory neurons in mouse visual and olfactory systems. Nat Struct Mol Biol 26:297-307.
Tiwari P, Sahay S, Pandey M, Qadri S S, Gupta K P (2015) Combinatorial chemopreventive effect of butyric acid, nicotinamide and calcium glucarate against the 7,12-dimethylbenz(a)anthracene induced mouse skin tumorigenesis attained by enhancing the induction of intrinsic apoptotic events. Chem Biol Interact 226:1-11.
Todd L, Zelinka C (2017) Valproic Acid for a Treatment of Retinitis Pigmentosa: Reasons for Optimism and Caution. J Neurosci 37:5215-5217.
Totan Y, Güler E, Yüce A, Dervişogullari M S (2017) The adverse effects of valproic acid on visual functions in the treatment of retinitis pigmentosa. Indian J Ophthalmol 65:984-988.
Trachsel-Moncho L, Benlloch-Navarro S, Fernández-Carbonell Á, Ramírez-Lamelas D T, Olivar T, Silvestre D, Poch E, Miranda M (2018) Oxidative stress and autophagy-related changes during retinal degeneration and development. Cell Death Dis 9:812.
Vent-Schmidt R Y J, Wen R H, Zong Z, Chiu C N, Tam B M, May C G, Moritz O L (2017) Opposing Effects of Valproic Acid Treatment Mediated by Histone Deacetylase Inhibitor Activity in Four Transgenic X. laevis Models of Retinitis Pigmentosa. J Neurosci 37:1039-1054.
Wang T, Reingruber J, Woodruff M L, Majumder A, Camarena A, Artemyev N O, Fain G L, Chen J (2018) The PDE6 mutation in the rd10 retinal degeneration mouse model causes protein mislocalization and instability and promotes cell death through increased ion influx. J Biol Chem 293:15332-15346.
Whyte W A, Bilodeau S, Orlando D A, Hoke H A, Frampton G M, Foster C T, Cowley S M, Young R A (2012) Enhancer decommissioning by LSD1 during embryonic stem cell differentiation. Nature 482:221-225.
Wieghofer P, Hagemeyer N, Sankowski R, Schlecht A, Staszewski O, Amann L, Gruber M, Koch J, Hausmann A, Zhang P, Boneva S, Masuda T, Hilgendorf I, Goldmann T, Böttcher C, Priller J, Rossi F M, Lange C, Prinz M (2021) Mapping the origin and fate of myeloid cells in distinct compartments of the eye by single-cell profiling. Embo j 40:e105123.
Won J, Shi L Y, Hicks W, Wang J, Hurd R, Naggert J K, Chang B, Nishina P M (2011) Mouse model resources for vision research. J Ophthalmol 2011:391384.
Yoshida N, Ikeda Y, Notomi S, Ishikawa K, Murakami Y, Hisatomi T, Enaida H, Ishibashi T (2013) Laboratory evidence of sustained chronic inflammatory reaction in retinitis pigmentosa. Ophthalmology 120:e5-12.
Zabel M K, Zhao L, Zhang Y, Gonzalez S R, Ma W, Wang X, Fariss R N, Wong W T (2016) Microglial phagocytosis and activation underlying photoreceptor degeneration is regulated by CX3CL1-CX3CR1 signaling in a mouse model of retinitis pigmentosa. Glia 64:1479-1491.
Zencak D, Schouwey K, Chen D, Ekström P, Tanger E, Bremner R, van Lohuizen M, Arsenijevic Y (2013) Retinal degeneration depends on Bmi1 function and reactivation of cell cycle proteins. Proc Natl Acad Sci USA 110:E593-601.
Zhang H, Dai X, Qi Y, He Y, Du W, Pang J J (2015) Histone Deacetylases Inhibitors in the Treatment of Retinal Degenerative Diseases: Overview and Perspectives. J Ophthalmol 2015:250812.
Zhao L, Zabel M K, Wang X, Ma W, Shah P, Fariss R N, Qian H, Parkhurst C N, Gan W B, Wong W T (2015) Microglial phagocytosis of living photoreceptors contributes to inherited retinal degeneration. EMBO Mol Med 7:1179-1197.
Zheng S, Xiao L, Liu Y, Wang Y, Cheng L, Zhang J, Yan N, Chen D (2018) DZNep inhibits H3K27me3 deposition and delays retinal degeneration in the rd1 mice. Cell Death Dis 9:310.

What is claimed is:

1. A method of treating or preventing a retinal disease or disorder in a subject, the method comprising administering to the subject a composition comprising an epigenetic modifier and a pharmaceutically acceptable carrier, wherein the epigenetic modifier comprises an inhibitor of chromatin modifying enzymes.

2. The method of claim 1, wherein the inhibitor comprises a demethylase inhibitor, a methyltransferase inhibitor, a deacetylase inhibitor, or variants thereof.

3. The method of claim 2, wherein the demethylase inhibitor comprises a lysine-specific demethylase 1 (LSD1) inhibitor.

4. The method of claim 3, wherein the LSD1 inhibitor comprises tranylcypromine (TCP), GSK2879552, or variants thereof.

5. The method of claim 2, wherein the methyltransferase inhibitor comprises a histone methyltransferase inhibitor.

6. The method of claim 5, wherein the histone methyltransferase inhibitor comprises 3-deazaneplanocin A (DZNep), UNC0642, or variants thereof.

7. The method of claim 2, wherein the deacetylase inhibitor comprises a histone deacetylase 1 (HDAC) inhibitor.

8. The method of claim 7, wherein the HDAC1 inhibitor comprises romidepsin, or variants thereof.

9. The method of claim 1, wherein the composition is administered for at least 14 days.

10. The method of claim 1, wherein the composition is administered by a method selected from the group consisting of administration as an eye drop, administration by an intraocular injection, administration as a gel to an eye of the subject, administration as an implant in the eye that releases the epigenetic modifier over time, administration as an expression vector that expresses the epigenetic modifier, and administration using a cell-based expression system.

11. The method of claim 1, wherein the pharmaceutically acceptable carrier comprises a saline solution, a gelatin composition, an excipient, a diluent, a salt, a buffer, a stabilizer, a lipid, an emulsion, or a nanoparticle.

12. The method of claim 1, wherein the method comprises administering an additional therapeutic agent to the subject, wherein the therapeutic agent comprises an antibiotic, an anesthetic, a sedative, an anti-inflammatory composition, or a hydrating solution.

13. The method of claim 1, wherein the epigenetic modifier decondenses a chromatin to increase or maintain expression of one or more genes selected from the group consisting of CRX, NRL, RHO, PRPH2, NR2E3, PDE6B, SAG, ROM1, CNGA1, CNGB1, NEUROD1, PTP4A3, ABCA4, FAM83G, LEFTY2, SFRP5, and UPK1B.

14. The method of claim 1, wherein the epigenetic modifier alters the chromatin to decrease expression of one or more genes selected from the group consisting of GFAP, C1QB, C1QA, H2-AA, CX3CR1, PTPRC, CD74, CST7, and AIF1.

15. The method of claim 1, wherein the method reduces or prevents degeneration of a retinal cell.

16. The method of claim 1, wherein the method decreases inflammation, gliosis, or cell death in the subject.

17. The method of claim 1, wherein the method increases an anti-inflammatory response in the subject.

18. The method of claim 1, wherein the retinal disease comprises retinitis pigmentosa or macular degeneration.

19. The method of claim 1, wherein the subject is a mammal.

20. The method of claim 1, wherein the subject is a human.


SEQUENCE LISTING (108 sequences)

SEQ ID NO: 1 - DNA, 20 bp, Mus musculus
  source 1..20: mol_type = genomic DNA
tgctcttggcagaaagtctc

SEQ ID NO: 2 - DNA, 20 bp, Mus musculus
  source 1..20: mol_type = genomic DNA
gtacaccccctgaaaagcaa

SEQ ID NO: 3 - DNA, 20 bp, Mus musculus
  source 1..20: mol_type = genomic DNA
agcccgtgtttgtaatggag

SEQ ID NO: 4 - DNA, 23 bp, Mus musculus
  source 1..23: mol_type = genomic DNA
gagcgtgtgtgacagtattagcg

SEQ ID NO: 5 - DNA, 18 bp, Mus musculus
  source 1..18: mol_type = genomic DNA
gaggggagccaggagctg

SEQ ID NO: 6 - DNA, 20 bp, Mus musculus
  source 1..20: mol_type = genomic DNA
gctgatgaagacacagtggg

SEQ ID NO: 7 - DNA, 20 bp, Mus musculus
  source 1..20: mol_type = genomic DNA
ccccagttcattattggagg

SEQ ID NO: 8 - DNA, 20 bp, Mus musculus
  source 1..20: mol_type = genomic DNA
caggcccgtggacattggtt

SEQ ID NO: 9 - DNA, 20 bp, Mus musculus
  source 1..20: mol_type = genomic DNA
caaacctgtgagccagatgc

SEQ ID NO: 10 - DNA, 23 bp, Mus musculus
  source 1..23: mol_type = genomic DNA
aatacgtggcattccttcgtaaa

SEQ ID NO: 11 - DNA, 20 bp, Mus musculus
  source 1..20: mol_type = genomic DNA
agaggaggaacactactgcg

SEQ ID NO: 12 - DNA, 21 bp, Mus musculus
  source 1..21: mol_type = genomic DNA
cgaactacatgcaggaagacc

SEQ ID NO: 13 - DNA, 19 bp, Mus musculus
  source 1..19: mol_type = genomic DNA
cagcatcgaccggtacctt

SEQ ID NO: 14 - DNA, 24 bp, Mus musculus
  source 1..24: mol_type = genomic DNA
gtgagatctacagcttcatgaagg

SEQ ID NO: 15 - DNA, 25 bp, Mus musculus
  source 1..25: mol_type = genomic DNA
ggttgtacagcagttagagctacag

SEQ ID NO: 16 - DNA, 22 bp, Mus musculus
  source 1..22: mol_type = genomic DNA
gagagaaaggttgaatcgctgg

SEQ ID NO: 17 - DNA, 18 bp, Mus musculus
  source 1..18: mol_type = genomic DNA
accatgcctcctgagttg

SEQ ID NO: 18 - DNA, 24 bp, Mus musculus
  source 1..24: mol_type = genomic DNA
gtcttgactaagaggtcaaattcc

SEQ ID NO: 19 - DNA, 22 bp, Mus musculus
  source 1..22: mol_type = genomic DNA
ttccaagctagagaaggcagac

SEQ ID NO: 20 - DNA, 18 bp, Mus musculus
  source 1..18: mol_type = genomic DNA
aagctgctgctggagcag

SEQ ID NO: 21 - DNA, 19 bp, Mus musculus
  source 1..19: mol_type = genomic DNA
cctgtgacctttcccatgc

SEQ ID NO: 22 - DNA, 21 bp, Mus musculus
  source 1..21: mol_type = genomic DNA
cttcaaacctgaaacacgagg

SEQ ID NO: 23 - DNA, 26 bp, Mus musculus
  source 1..26: mol_type = genomic DNA
gtgcctccttcacccaccttcagtga

SEQ ID NO: 24 - DNA, 21 bp, Mus musculus
  source 1..21: mol_type = genomic DNA
cagcatccgcttcaactccaa

SEQ ID NO: 25 - DNA, 21 bp, Mus musculus
  source 1..21: mol_type = genomic DNA
tcgccacctctactttgatag

SEQ ID NO: 26 - DNA, 21 bp, Mus musculus
  source 1..21: mol_type = genomic DNA
ctgacgagtatgaggccaaag

SEQ ID NO: 27 - DNA, 23 bp, Mus musculus
  source 1..23: mol_type = genomic DNA
ccttatgtggtcagccaataaag

SEQ ID NO: 28 - DNA, 20 bp, Mus musculus
  source 1..20: mol_type = genomic DNA
tggatcagcaatcgctacct

SEQ ID NO: 29 - DNA, 22 bp, Mus musculus
  source 1..22: mol_type = genomic DNA
tacagagcttcctccaaggaaa

SEQ ID NO: 30 - DNA, 19 bp, Mus musculus
  source 1..19: mol_type = genomic DNA
tgcctcacctacacacacc

SEQ ID NO: 31 - DNA, 19 bp, Mus musculus
  source 1..19: mol_type = genomic DNA
ttgtgtggatgtcatctgc

SEQ ID NO: 32 - DNA, 21 bp, Mus musculus
  source 1..21: mol_type = genomic DNA
cttctccaacgtcacaggcgt

SEQ ID NO: 33 - DNA, 20 bp, Mus musculus
  source 1..20: mol_type = genomic DNA
agcagggctttgatggtagc

SEQ ID NO: 34 - DNA, 23 bp, Mus musculus
  source 1..23: mol_type = genomic DNA
ccgtcagaatgtgtgagaaccag

SEQ ID NO: 35 - DNA, 18 bp, Mus musculus
  source 1..18: mol_type = genomic DNA
gccatgagtgtcctcacc

SEQ ID NO: 36 - DNA, 18 bp, Mus musculus
  source 1..18: mol_type = genomic DNA
ctttctggctgtggcgag

SEQ ID NO: 37 - DNA, 20 bp, Mus musculus
  source 1..20: mol_type = genomic DNA
gtgaagaagctcaagaagac

SEQ ID NO: 38 - DNA, 21 bp, Mus musculus
  source 1..21: mol_type = genomic DNA
gagtggaaacttttgtccgag

SEQ ID NO: 39 - DNA, 29 bp, Mus musculus
  source 1..29: mol_type = genomic DNA
gttttccctctcgtccatcagaggacctg

SEQ ID NO: 40 - DNA, 18 bp, Mus musculus
  source 1..18: mol_type = genomic DNA
acggagctcccagaagac

SEQ ID NO: 41 - DNA, 19 bp, Mus musculus
  source 1..19: mol_type = genomic DNA
tgggcatcactttcactcc

SEQ ID NO: 42 - DNA, 20 bp, Mus musculus
  source 1..20: mol_type = genomic DNA
cagggtgggtcaccatctat

SEQ ID NO: 43 - DNA, 20 bp, Mus musculus
  source 1..20: mol_type = genomic DNA
cacagccttgattttgctga

SEQ ID NO: 44 - DNA, 22 bp, Mus musculus
  source 1..22: mol_type = genomic DNA
catgggattacacttggtctcg

SEQ ID NO: 45 - DNA, 18 bp, Mus musculus
  source 1..18: mol_type = genomic DNA
ggattgcctttcacgccc

SEQ ID NO: 46 - DNA, 19 bp, Mus musculus
  source 1..19: mol_type = genomic DNA
gctgttgatggtcctcagg

SEQ ID NO: 47 - DNA, 20 bp, Mus musculus
  source 1..20: mol_type = genomic DNA
gccaggatttcctcattcaa

SEQ ID NO: 48 - DNA, 21 bp, Mus musculus
  source 1..21: mol_type = genomic DNA
cagccgctcccgttgaagata

SEQ ID NO: 49 - DNA, 18 bp, Mus musculus
  source 1..18: mol_type = genomic DNA
ggtcctgggtcatgttgc

SEQ ID NO: 50 - DNA, 22 bp, Mus musculus
  source 1..22: mol_type = genomic DNA
gagccattgtcatcgtcagaaa

SEQ ID NO: 51 - DNA, 22 bp, Mus musculus
  source 1..22: mol_type = genomic DNA
aagtaatccatgaggagccaga

SEQ ID NO: 52 - DNA, 20 bp, Mus musculus
  source 1..20: mol_type = genomic DNA
cactggcagaggagaacagg

SEQ ID NO: 53 - DNA, 19 bp, Mus musculus
  source 1..19: mol_type = genomic DNA
gctgcactgtccggttgtt

SEQ ID NO: 54 - DNA, 22 bp, Mus musculus
  source 1..22: mol_type = genomic DNA
tgagatgagcttgtccaactcc

SEQ ID NO: 55 - DNA, 23 bp, Mus musculus
  source 1..23: mol_type = genomic DNA
ggagtatgaggtaagctctgtgg

SEQ ID NO: 56 - DNA, 20 bp, Mus musculus
  source 1..20: mol_type = genomic DNA
cggcgatagtcgttagcttc

SEQ ID NO: 57 - DNA, 19 bp, Mus musculus
  source 1..19: mol_type = genomic DNA
tgactctggatcgaagcac

SEQ ID NO: 58 - DNA, 20 bp, Mus musculus
  source 1..20: mol_type = genomic DNA
ttctgagccatgtgatgttg

SEQ ID NO: 59 - DNA, 18 bp, Mus musculus
  source 1..18: mol_type = genomic DNA
gcacctcggtgttaacgc

SEQ ID NO: 60 - DNA, 19 bp, Mus musculus
  source 1..19: mol_type = genomic DNA
gcagcttcatctgcgtgtc

SEQ ID NO: 61 - DNA, 19 bp, Mus musculus
  source 1..19: mol_type = genomic DNA
agaagtgctaaggcaacgc

SEQ ID NO: 62 - DNA, 21 bp, Mus musculus
  source 1..21: mol_type = genomic DNA
cctcaaagatgggagcaggag

SEQ ID NO: 63 - DNA, 25 bp, Mus musculus
  source 1..25: mol_type = genomic DNA
gcgtgcggcgcctctgcttcagccg

SEQ ID NO: 64 - DNA, 23 bp, Mus musculus
  source 1..23: mol_type = genomic DNA
gcagatgagggaaagaggaatga

SEQ ID NO: 65 - DNA, 18 bp, Mus musculus
  source 1..18: mol_type = genomic DNA
agccgcattggacgttag

SEQ ID NO: 66 - DNA, 21 bp, Mus musculus
  source 1..21: mol_type = genomic DNA
taggcagagtccgtatgcagt

SEQ ID NO: 67 - DNA, 20 bp, Mus musculus
  source 1..20: mol_type = genomic DNA
ccatctggagtctttggtcc

SEQ ID NO: 68 - DNA, 20 bp, Mus musculus
  source 1..20: mol_type = genomic DNA
ctgtagtaattcagcagagc

SEQ ID NO: 69 - DNA, 17 bp, Mus musculus
  source 1..17: mol_type = genomic DNA
cacggtgttgggaacgg

SEQ ID NO: 70 - DNA, 25 bp, Mus musculus
  source 1..25: mol_type = genomic DNA
acatgagtcattagacacactgatg

SEQ ID NO: 71 - DNA, 21 bp, Mus musculus
  source 1..21: mol_type = genomic DNA
gaagtgtgtgtgatgaacagg

SEQ ID NO: 72 - DNA, 20 bp, Mus musculus
  source 1..20: mol_type = genomic DNA
ggaccacagggcgatttcac

SEQ ID NO: 73 - DNA, 21 bp, Mus musculus
  source 1..21: mol_type = genomic DNA
ttcagctcatccttggccttc

SEQ ID NO: 74 - DNA, 23 bp, Mus musculus
  source 1..23: mol_type = genomic DNA
atcctcccgaactttacagcatc

SEQ ID NO: 75 - DNA, 17 bp, Mus musculus
  source 1..17: mol_type = genomic DNA
ggcatgctgcactttcc

SEQ ID NO: 76 - DNA, 19 bp, Mus musculus
  source 1..19: mol_type = genomic DNA
gccatgtagaagacacggc

SEQ ID NO: 77 - DNA, 21 bp, Mus musculus
  source 1..21: mol_type = genomic DNA
actggaggtagaactggcgtc

SEQ ID NO: 78 - DNA, 25 bp, Mus musculus
  source 1..25: mol_type = genomic DNA
gaagcgtgtacttatccttcttcat

SEQ ID NO: 79 - DNA, 25 bp, Mus musculus
  source 1..25: mol_type = genomic DNA
gcttccgcttggctagcctcttgct

SEQ ID NO: 80 - DNA, 18 bp, Mus musculus
  source 1..18: mol_type = genomic DNA
ccatccttggcagacttg

SEQ ID NO: 81 - DNA, 20 bp, Mus musculus
  source 1..20: mol_type = genomic DNA
agctgcgggtatctccataa

SEQ ID NO: 82 - DNA, 21 bp, Mus musculus
  source 1..21: mol_type = genomic DNA
ttagaataaagcggttccacc

SEQ ID NO: 83 - DNA, 19 bp, Mus musculus
  source 1..19: mol_type = genomic DNA
gctcaggttggcctcagac

SEQ ID NO: 84 - DNA, 21 bp, Mus musculus
  source 1..21: mol_type = genomic DNA
ggactttgatcctgctgcaag

SEQ ID NO: 85 - DNA, 23 bp, Mus musculus
  source 1..23: mol_type = genomic DNA
agatagaaggtgcagcaagaagg

SEQ ID NO: 86 - DNA, 24 bp, Mus musculus
  source 1..24: mol_type = genomic DNA
gaacagagactgcagagacagatc

SEQ ID NO: 87 - DNA, 20 bp, Mus musculus
  source 1..20: mol_type = genomic DNA
gcctggccacaaaagaaata

SEQ ID NO: 88 - DNA, 21 bp, Mus musculus
  source 1..21: mol_type = genomic DNA
cacggtcacttctccacagcg

SEQ ID NO: 89 - DNA, 18 bp, Mus musculus
  source 1..18: mol_type = genomic DNA
caggacccgtttcatcag

SEQ ID NO: 90 - DNA, 21 bp, Mus musculus
  source 1..21: mol_type = genomic DNA
caggcaagctacagacctcag

SEQ ID NO: 91 - DNA, 20 bp, Mus musculus
  source 1..20: mol_type = genomic DNA
ggaattcccagaggactctg

SEQ ID NO: 92 - DNA, 20 bp, Mus musculus
  source 1..20: mol_type = genomic DNA
tgaagcccacacaggaagtg

SEQ ID NO: 93 - DNA, 25 bp, Mus musculus
  source 1..25: mol_type = genomic DNA
aaagcactctagtaaaagcaagtcc

SEQ ID NO: 94 - DNA, 23 bp, Mus musculus
  source 1..23: mol_type = genomic DNA
ttctgctgctgttcctcatttac

SEQ ID NO: 95 - DNA, 20 bp, Mus musculus
  source 1..20: mol_type = genomic DNA
aggctgagatcctaggcaga

SEQ ID NO: 96 - DNA, 20 bp, Mus musculus
  source 1..20: mol_type = genomic DNA
ttcggagctacagtggaatc

SEQ ID NO: 97 - DNA, 22 bp, Mus musculus
  source 1..22: mol_type = genomic DNA
ccacactagtgcgagacctgag

SEQ ID NO: 98 - DNA, 22 bp, Mus musculus
  source 1..22: mol_type = genomic DNA
caagggttctaggaaatggtcc

SEQ ID NO: 99 - DNA, 22 bp, Mus musculus
  source 1..22: mol_type = genomic DNA
agttgtaactccaaaccttgcg

SEQ ID NO: 100 - DNA, 24 bp, Mus musculus
  source 1..24: mol_type = genomic DNA
aatcttgatggtgtctgatattgg

SEQ ID NO: 101 - DNA, 20 bp, Mus musculus
  source 1..20: mol_type = genomic DNA
cccaaactttctttgccaca

SEQ ID NO: 102 - DNA, 20 bp, Mus musculus
  source 1..20: mol_type = genomic DNA
ccccattcccctttgcactg

SEQ ID NO: 103 - DNA, 18 bp, Mus musculus
  source 1..18: mol_type = genomic DNA
ggtgttcctgctgctgtg

SEQ ID NO: 104 - DNA, 23 bp, Mus musculus
  source 1..23: mol_type = genomic DNA
taatatgcaggaatccttcatgg

SEQ ID NO: 105 - DNA, 19 bp, Mus musculus
  source 1..19: mol_type = genomic DNA
ctcttcgtagacagagacc

SEQ ID NO: 106 - DNA, 25 bp, Mus musculus
  source 1..25: mol_type = genomic DNA
ctataagcatccatatgcactgtgg

SEQ ID NO: 107 - DNA, 23 bp, Mus musculus
  source 1..23: mol_type = genomic DNA
ggtgtactattacgctcaacacg

SEQ ID NO: 108 - DNA, 24 bp, Mus musculus
  source 1..24: mol_type = genomic DNA
ctaataacgatggtattggctcag